(19) United States
(12) Patent Application Publication
(10) Pub. No.: US 2011/0250544 A1
(43) Pub. Date: Oct. 13, 2011

(54) BOTTOM ANTIREFLECTIVE COATING COMPOSITIONS

(75) Inventors: Weihong Liu, Bridgewater, NJ (US); Guanyang Lin, Whitehouse Station, NJ (US); JoonYeon Cho, Bridgewater, NJ (US); Jian Yin, Bridgewater, NJ (US); Salem K. Mullen, Florham Park, NJ (US); Mark Neisser, Whitehouse Station, NJ (US)

(73) Assignee: AZ ELECTRONIC MATERIALS USA CORP., SOMERVILLE, NJ (US)

(21) Appl. No.: 13/162,065

(22) Filed: Jun. 16, 2011

Related U.S. Application Data
(62) Division of application No. 12/250,563, filed on Oct. 14, 2008, now abandoned.

(51) Int. Cl. G03F 7/20; C07D 251/04; C09D 5/00

(52) U.S. Cl. 430/325; 252/582; 544/221

(57) ABSTRACT

Antireflective coating compositions are discussed.

### CROSS-REFERENCE TO RELATED APPLICATION

This application is a divisional of U.S. patent application Ser. No. 12/250,563 filed Oct. 14, 2008, the contents of which are hereby incorporated herein by reference.

### FIELD OF INVENTION

The present invention relates to novel coating compositions and their use in image processing by forming a thin layer of the novel coating composition between a reflective substrate and a photoresist coating. Such compositions are particularly useful in the fabrication of semiconductor devices by photolithographic techniques. The invention further relates to a polymer for the coating composition.

### BACKGROUND

Photoresist compositions are used in microlithography processes for making miniaturized electronic components such as in the fabrication of computer chips and integrated circuits. Generally, in these processes, a thin coating of film of a photoresist composition is first applied to a substrate material, such as silicon wafers used for making integrated circuits. The coated substrate is then baked to evaporate any solvent in the photoresist composition and to fix the coating onto the substrate. The baked coated surface of the substrate is next subjected to an image-wise exposure to radiation.

This radiation exposure causes a chemical transformation in the exposed areas of the coated surface. Visible light, ultraviolet (UV) light, electron beam and X-ray radiant energy are radiation types commonly used today in microlithographic processes. After this image-wise exposure, the coated substrate is treated with a developer solution to dissolve and remove either the radiation-exposed or the unexposed areas of the photoresist.

The trend towards the miniaturization of semiconductor devices has led to the use of new photoresists that are sensitive to lower and lower wavelengths of radiation and has also led to the use of sophisticated multilevel systems to overcome difficulties associated with such miniaturization.

High resolution, chemically amplified, deep ultraviolet (100-300 nm) positive and negative tone photoresists are available for patterning images with less than quarter micron geometries. There are two major deep ultraviolet (uv) exposure technologies that have provided significant advancement in miniaturization, and these are lasers that emit radiation at 248 nm and 193 nm. Examples of such photoresists are given in the following patents and incorporated herein by reference, U.S. Pat. No. 4,491,628, U.S. Pat. No. 5,350,660, EP 794458 and GB 2320718. Photoresists for 248 nm have typically been based on substituted polyhydroxystyrene and its copolymers. On the other hand, photoresists for 193 nm exposure require non-aromatic polymers, since aromatics are opaque at this wavelength. Generally, alicyclic hydrocarbons are incorporated into the polymer to replace the etch resistance lost by not having aromatics present. Furthermore, at lower wavelengths the reflection from the substrate becomes increasingly detrimental to the lithographic performance of the photoresist. Therefore, at these wavelengths antireflective coatings become critical.

The use of highly absorbing antireflective coatings in photolithography is a simpler approach to diminish the problems that result from back reflection of light from highly reflective substrates. Two major disadvantages of back reflectivity are thin film interference effects and reflective notching. Thin film interference, or standing waves, result in changes in critical line width dimensions caused by variations in the total light intensity in the resist film as the thickness of the resist changes. Reflective notching becomes severe as the photoresist is patterned over substrates containing topographical features, which scatter light through the photoresist film, leading to line width variations, and in the extreme case, forming regions with complete photoresist loss.

In the past dyed photoresists have been utilized to solve these reflectivity problems. However, it is generally known that dyed resists only reduce reflectivity from the substrate but do not substantially eliminate it. In addition, dyed resists also cause reduction in the lithographic performance of the photoresist, together with possible sublimation of the dye and incompatibility of the dye in resist films.

In cases where further reduction or elimination of line width variation is required, the use of bottom antireflective coating provides the best solution for the elimination of reflectivity. The bottom antireflective coating is applied to the substrate prior to coating with the photoresist and prior to exposure. The resist is exposed imagewise and developed. The antireflective coating in the exposed area is then etched, typically in an oxygen plasma, and the resist pattern is thus transferred to the substrate. The etch rate of the antireflective film should be relatively high in comparison to the photoresist so that the antireflective film is etched without excessive loss of the resist film during the etch process. Inorganic types of antireflective coatings include such films as TiN, TiON, TiW and spin-on organic polymer in the range of 30 nm. Inorganic B.A.R.C.s require precise control of the film thickness, uniformity of film, special deposition equipment, complex adhesion promotion techniques prior to resist coating, separate dry etching pattern transfer step, and dry etching for removal.

Organic B.A.R.C.s are more preferred and have been formulated by adding dyes to a polymer coating (Proc. SPIE, Vol. 1086 (1989), p. 106). Problems of such dye blended coatings include 1) separation of the polymer and dye components during spin coating 2) dye stripping into resist solvents, and 3) thermal diffusion into the resist upon the baking process. All these effects cause degradation of photoresist properties and therefore are not the preferred composition.

Light absorbing, film forming polymers are another option. Polymeric organic antireflective coatings are known in the art as described in EP 583,205, and incorporated herein by reference. However, these polymers have been found to be ineffective when used as antireflective coatings for photoresists sensitive to 193 nm. It is believed that such antireflective polymers are very aromatic in nature and thus are too reflective, acting as a mirror rather than absorbers. Additionally, these polymers being highly aromatic, have too low a dry etch rate, relative to the new type of non-aromatic photoresists used for 193 nm exposure, and are therefore ineffective for imaging and etching. Photoresist patterns may be damaged or may not be transferred exactly to the substrate if the dry etch rate of the antireflective coating is similar to or less than the etch rate of the photoresist coated on top of the antireflective coating.

Thinner photoresist film thickness will be used for maximum lithographic resolution and process latitude. Due to less resist film available for pattern transfer to underneath substrates through etching process, higher etch rate and thinner bottom antireflective coating (BARC) film thickness are required. To maintain good reflectivity control, thinner BARC film thickness will naturally require materials with higher real refractive index. In addition, for second generation of immersion lithography using high refractive index immersion fluid, both high refractive index photoresist and BARC materials are necessary.

### SUMMARY OF THE INVENTION

The present invention relates to an antireflective coating composition comprising a) a compound having the formula

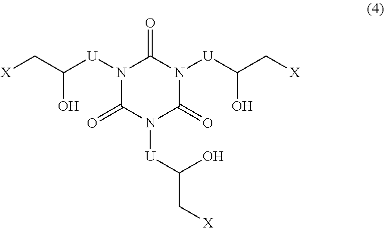

where X is selected from

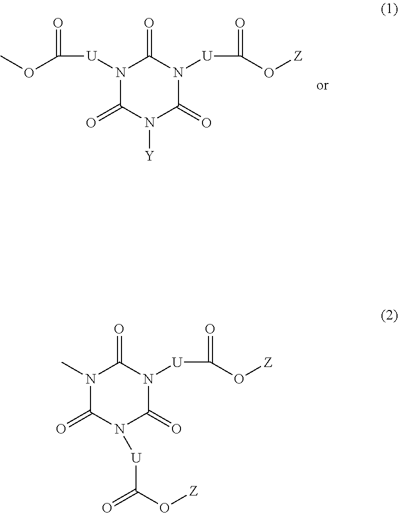

where U is a divalent linking group; Y is hydrogen or Z; and Z is the residue of an aromatic epoxide or aliphatic epoxide; and b) an acid or acid generator. Examples of the divalent linking group include an alkylene group, a phenylene group, a cycloalkylene group, etc. The composition can additionally contain a thermal acid generator and/or a crosslinker.

The invention also relates to a compound having the formula

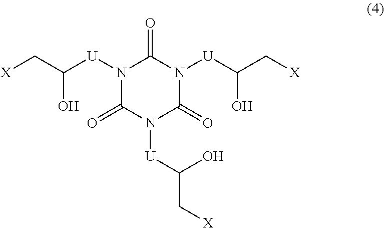

where X is selected from

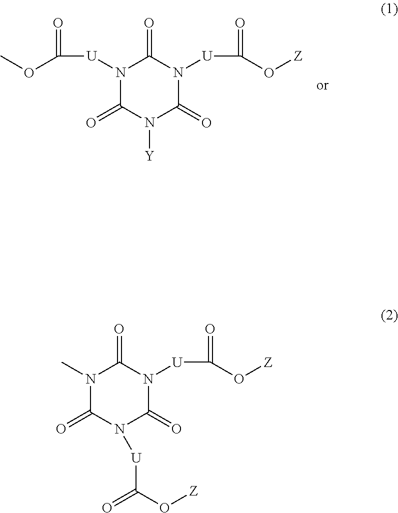

where U is a divalent linking group; Y is hydrogen or Z; and Z is the residue of an aromatic epoxide or aliphatic epoxide. Examples of the divalent linking group include an alkylene group, a phenylene group, a cycloalkylene group, etc.

The invention also relates to a compound having the formula

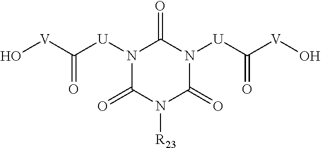

where U is a divalent linking group; V is a direct bond, C₁-C₁₀ straight or branched alkylene, or cycloalkylene group; and R₂₃ is hydrogen or C₁-C₁₀ alkyl.

The invention also relates to the reaction product of a compound having the formula

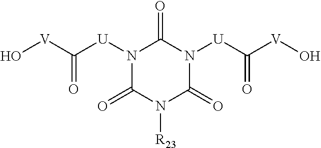

where U, V, and R₂₃ are described above with a polyhydroxy compound.

The invention also relates to a compound having a repeating unit selected from

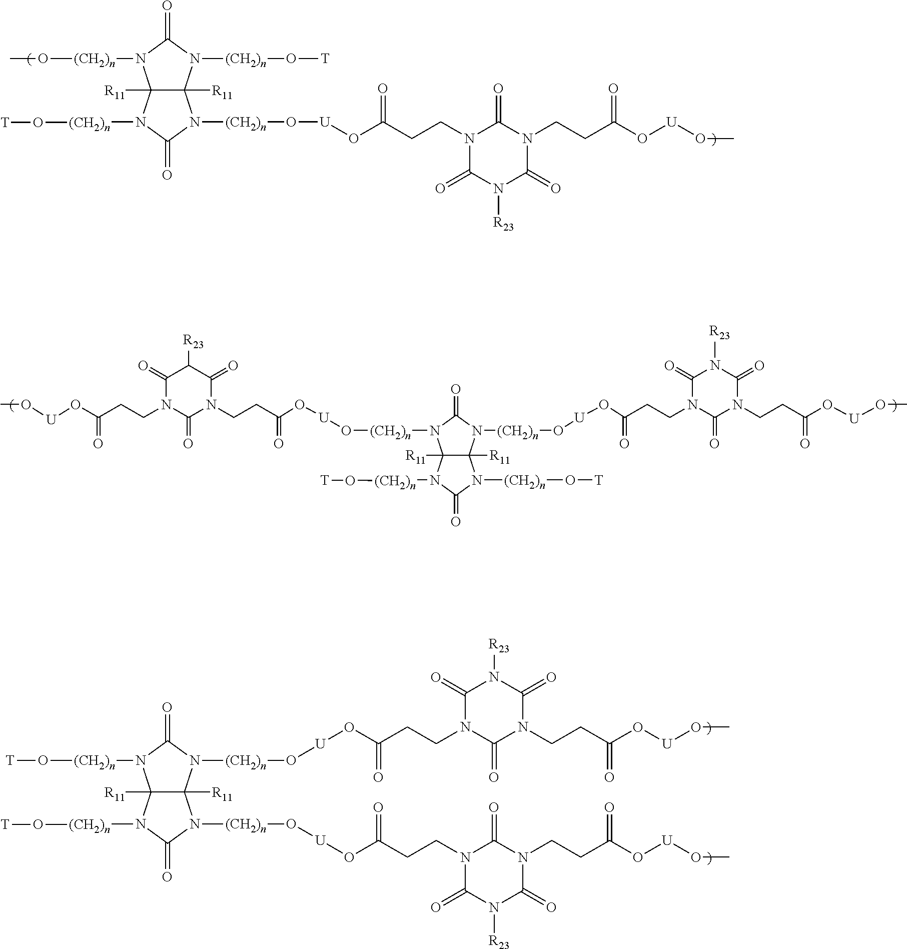

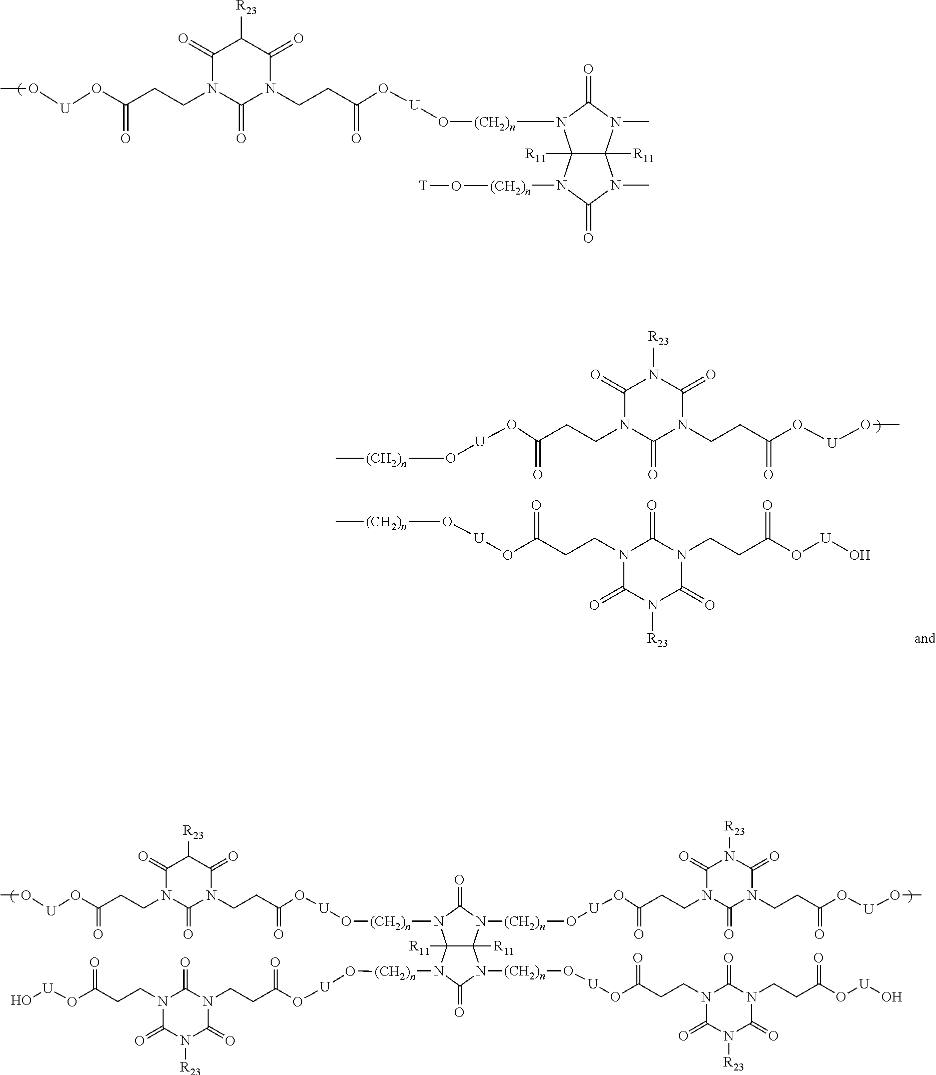

where U is a divalent linking group, each R₁₁ is hydrogen or C₁-C₁₀ alkyl, T is hydrogen, a straight or branched C₁-C₁₀ alkyl, or the residue of a polyhydroxy compound, R₂₃ is hydrogen or C₁-C₁₀ alkyl; and n is 0 to 4.

The invention also relates to a coated substrate comprising a substrate having thereon an antireflective coating layer formed from the antireflective coating composition described herein above where the antireflective coating layer has an absorption parameter (k) in the range of 0.01≦k<0.50 when measured at 193 nm.

The invention also relates to a process for forming an image comprising, a) coating and baking a substrate with the antireflective coating composition described hereinabove; b) coating and baking a photoresist film on top of the antireflective coating; c) imagewise exposing the photoresist; d) developing an image in the photoresist; e) optionally, baking the substrate after the exposing step.

### DETAILED DESCRIPTION OF THE INVENTION

The present invention relates to an antireflective coating composition comprising a) a compound having the formula

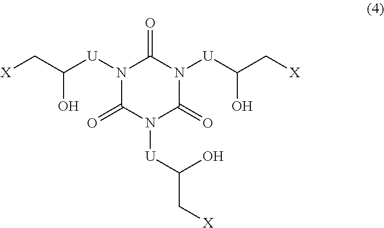

where X is selected from

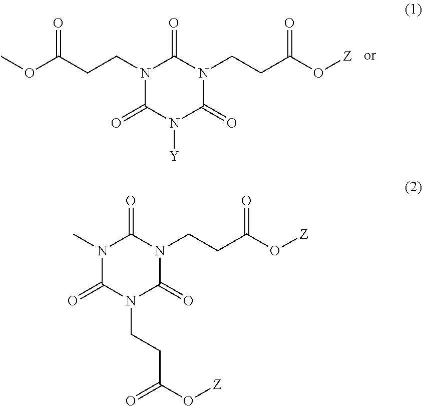

where Y is hydrogen or Z; and Z is the residue of an aromatic epoxide or aliphatic epoxide; and b) an acid or acid generator. The composition can additionally contain a thermal acid generator and/or a crosslinker.

The invention also relates to a compound having the formula

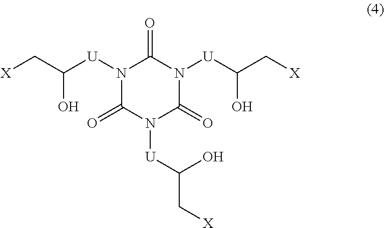

where X is selected from

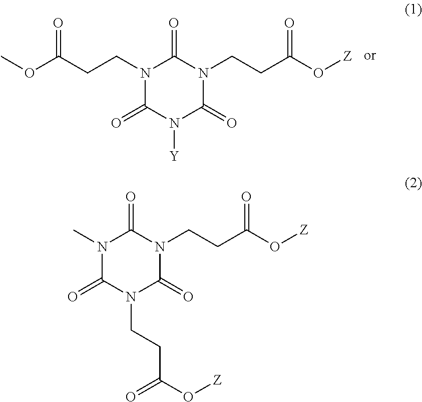

where Y is hydrogen or Z; and Z is the residue of an aromatic epoxide or aliphatic epoxide.

The invention also relates to the reaction product of a compound having the formula

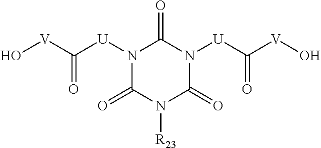

where U, V, and R₂₃ are described above with a polyhydroxy compound.

The invention also relates to a compound having a repeating unit selected from

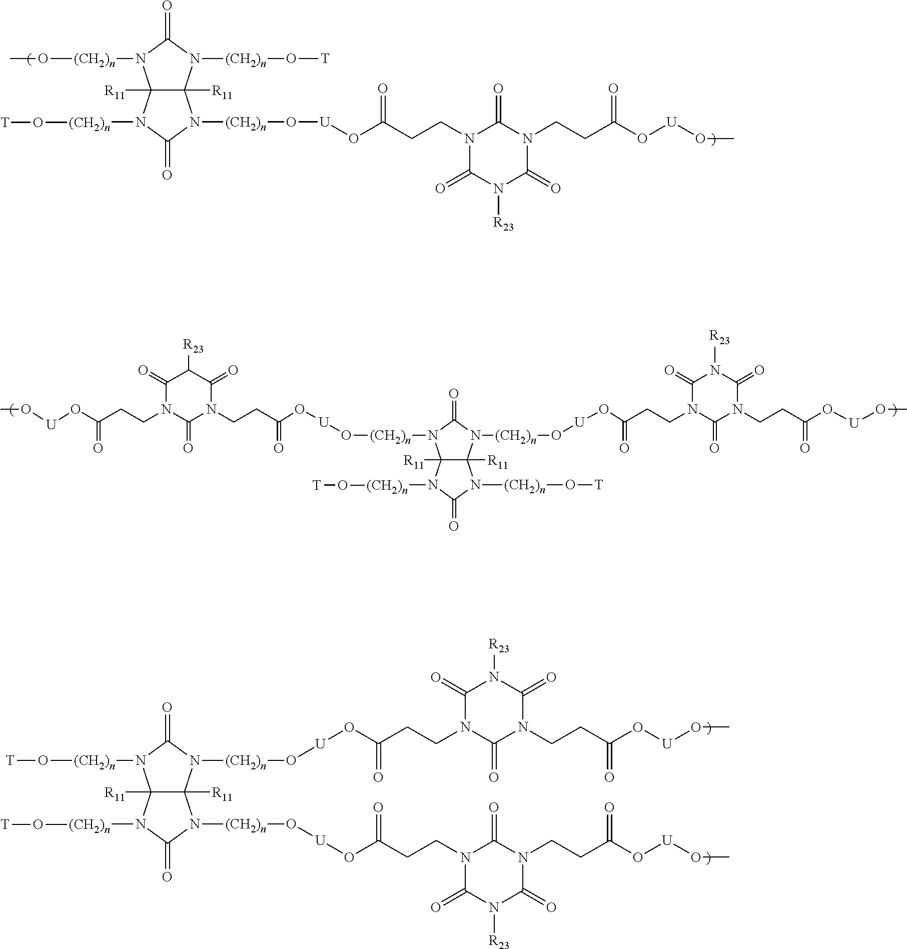

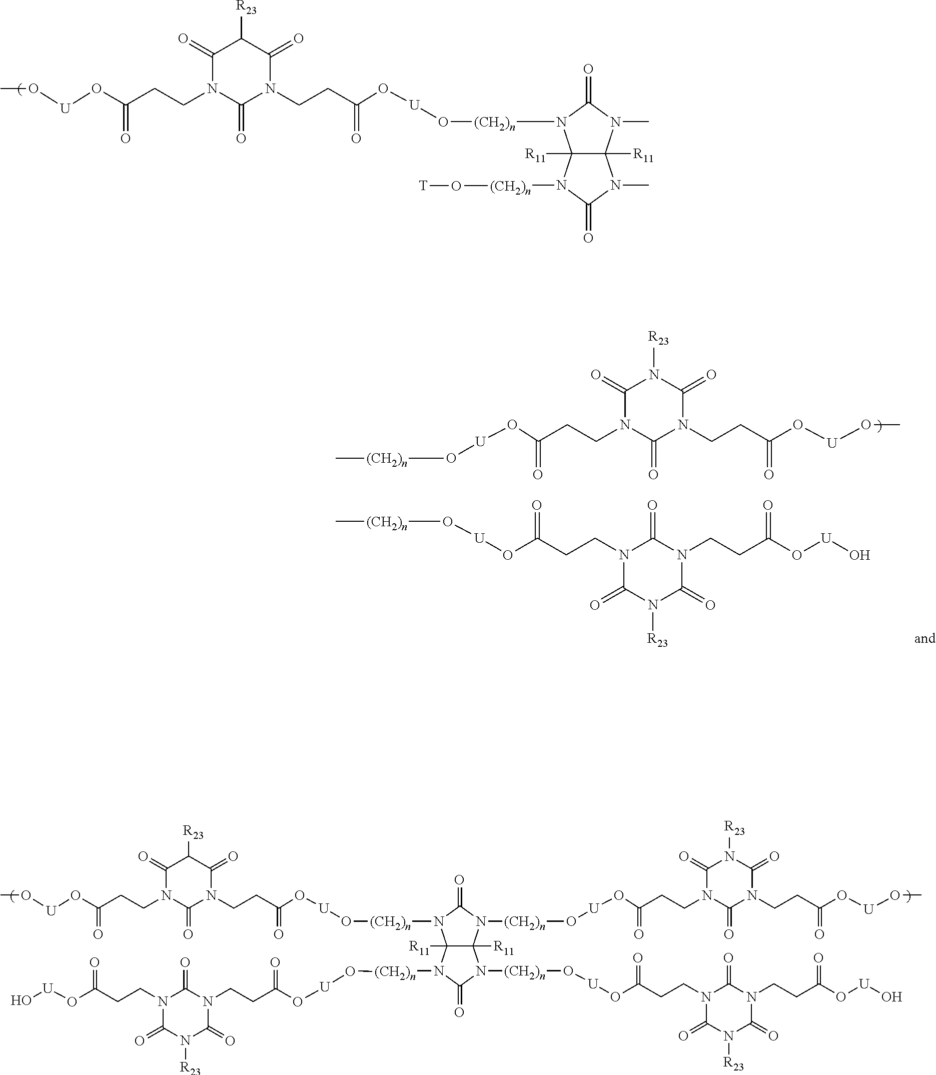

where U is a divalent linking group, each R₁₁ is hydrogen or C₁-C₁₀ alkyl, T is hydrogen, a straight or branched C₁-C₁₀ alkyl, or the residue of a polyhydroxy compound, R₂₃ is hydrogen or C₁-C₁₀ alkyl; and n is 0 to 4. Examples of the divalent linking group include an alkylene group, a phenylene group, a cycloalkylene group, etc.

The invention also relates to a compound having the formula

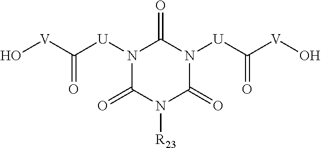

where U is a divalent linking group; V is a direct bond, C₁-C₁₀ straight or branched alkylene, or cycloalkylene group; and R₂₃ is hydrogen or C₁-C₁₀ alkyl. Examples of the divalent linking group include an alkylene group, a phenylene group, a cycloalkylene group, etc.

The invention also relates to a coated substrate comprising a substrate having thereon an antireflective coating layer formed from the antireflective coating composition described herein above where the antireflective coating layer has an absorption parameter (k) in the range of 0.01≦k<0.50 when measured at 193 nm.

The invention also relates to a process for forming an image comprising, a) coating and baking a substrate with the antireflective coating composition described hereinabove; b) coating and baking a photoresist film on top of the antireflective coating; c) imagewise exposing the photoresist; d) developing an image in the photoresist; e) optionally, baking the substrate after the exposing step.

The antireflective coating composition of the present invention first comprises a compound having the formula

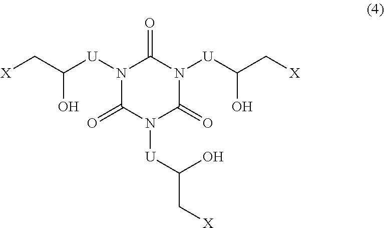

where X is selected from

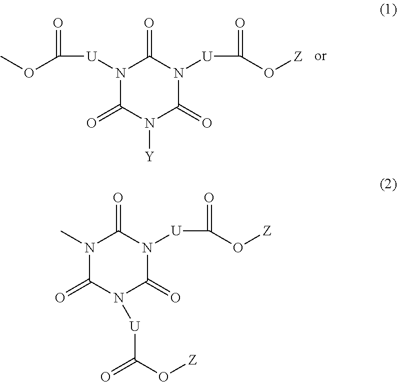

where U is a divalent linking group; Y is hydrogen or Z; and Z is the residue of an aromatic epoxide or aliphatic epoxide.

The compound (4) can be made by reacting a tris epoxy isocyanurate compound, for example, tris(2,3-expoypropyl)isocyanrate with the reaction product of bis(carboxyalkyl)isocyanurate and an aromatic or aliphatic oxide. The reaction of the bis(carboxyalkyl)isocyanurate and aromatic or aliphatic oxide is usually done in the presence of a catalyst, for example, beznyltriethylammonium chloride.

An example of the bis(carboxyethyl)isocyanurate includes bis(2-carboxyethyl)isocyanurate.

Examples of aromatic oxides include: styrene oxide, 1,2-epoxy-phenoxypropane, glycidyl-2-methylphenyl ether, (2,3-epoxypropyl)benzene, 1-phenylpropylene oxide, stilbene oxide, 2- (or 3- or 4-)halo(chloro, fluoro, bromo, iodo) stilbene oxide, benzyl glycidyl ether, C₁₋₁₀ straight or branched chain alkyl (e.g., methyl, ethyl, propyl, butyl, sec-butyl, tert-butyl, pentyl, hexyl, heptyl, and the like etc) phenyl glycidyl ether, 4-halo(chloro, fluoro, bromo, iodo)phenyl glycidyl ether, glycidyl 4-C₁₋₁₀ straight or branched chain alkoxy (e.g., methoxy, ethoxy, propoxy, butoxy, hexyloxy, heptyloxy, and the like etc) phenyl ether, 2,6-dihalo(chloro, fluoro, bromo, iodo)benzylmethyl ether, 3,4-dibenzyloxybenzyl halide (chloride, fluoride, bromide, iodide), 2-(or 4-)methoxybiphenyl, 3,3′-(or 4,4′-)diC₁₋₁₀ straight or branched chain alkoxy (e.g., methoxy, ethoxy, propoxy, butoxy, hexyloxy, heptyloxy, and the like etc) biphenyl, 4,4′-dimethoxyoctafluorobiphenyl, 1-(or 2-)C₁₋₁₀ straight or branched chain alkoxy (e.g., methoxy, ethoxy, propoxy, butoxy, hexyloxy, heptyloxy, and the like etc) naphthalene, 2-halo(chloro, fluoro, bromo, iodo)-6-methoxynaphthalene, 2,6-diC₁₋₁₀ straight or branched chain alkoxy (e.g., methoxy, ethoxy, propoxy, butoxy, hexyloxy, heptyloxy, and the like etc) naphthalene, 2,7-diC₁₋₁₀ straight or branched chain alkoxy (e.g., methoxy, ethoxy, propoxy, butoxy, hexyloxy, heptyloxy, and the like etc) naphthalene, 1,2,3,4,5,6-hexahalo(chloro, fluoro, bromo, iodo)-7-C₁₀ straight or branched chain alkoxy (e.g., methoxy, ethoxy, propoxy, butoxy, hexyloxy, heptyloxy, and the like etc) naphthalene, 9,10-bis(4-C₁₋₁₀ straight or branched chain alkoxy (e.g., methoxy, ethoxy, propoxy, butoxy, hexyloxy, heptyloxy, and the like etc) phenyl)-anthracene, 2-C₁₋₁₀ straight or branched chain alkyl (e.g., methyl, ethyl, propyl, butyl, sec-butyl, tert-butyl, pentyl, hexyl, heptyl, and the like etc)-9,10-diC₁₋₁₀ straight or branched chain alkoxy (e.g., methoxy, ethoxy, propoxy, butoxy, hexyloxy, heptyloxy, and the like etc) anthracene, 9,10-bis(4-C₁₋₁₀ straight or branched chain alkoxy (e.g., methoxy, ethoxy, propoxy, butoxy, hexyloxy, heptyloxy, and the like etc) phenyl)-2-halo(chloro, fluoro, bromo, iodo)-anthracene, 2,3,6,7,10,11-hexamethoxytriphenylene, glycidyl-3-(pentadecadienyl)phenyl ether, 4-t-butylphenylglycidyl ether, triphenylolmethane triglycidyl ether, [(4-(1-heptyl-8-[3-(oxiranylmethoxy)phenyl]-octyl)phenoxy)methyl]oxirane, tetraphenylolethane tetraglycidyl ether, hydroxyphenol diglycidyl ether, etc.

Examples of aliphatic oxides include ethylene oxide, propylene oxide, butylene oxides, including isobutylene oxide, 1,2-butylene oxide and 2,3-butylene oxide, pentylene oxide, cyclohexene oxide, decyl glycidyl ether, and dodecyl glycidyl ether.

The bis(carboxyalkyl)isocyanurate is typically reacted with the aromatic or aliphatic oxide in an about 1:1 mol ratio. The resulting reaction product is then typically reacted with the tris epoxy isocyanurate compound in an about 3:1 mol ratio.

Examples of (4) include

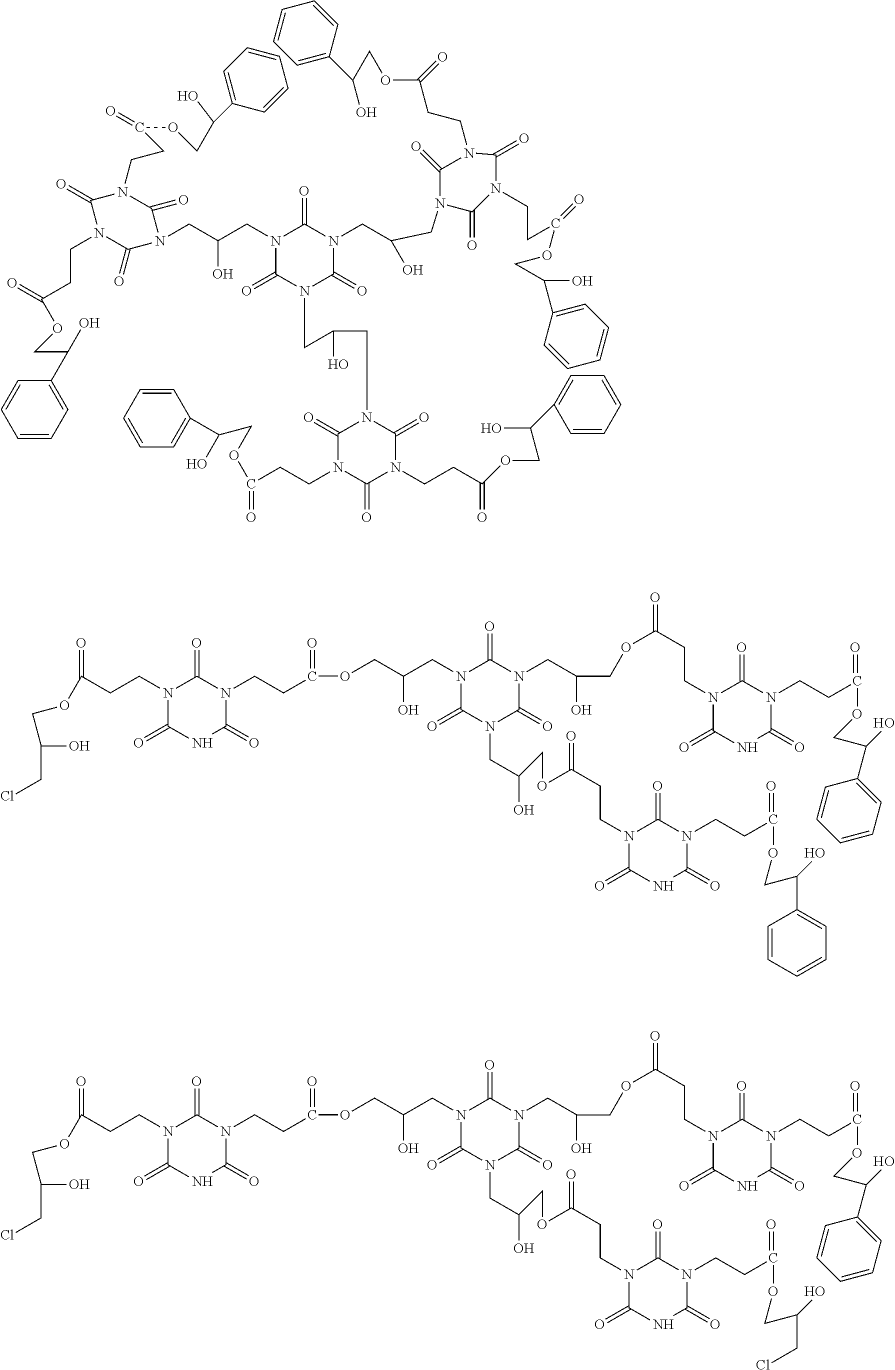

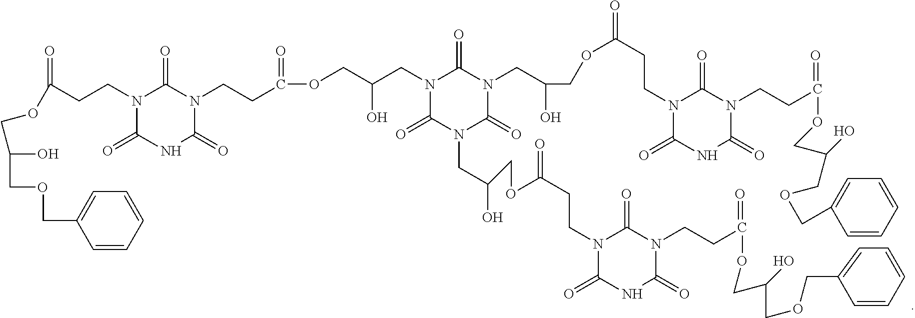

The acid generator used with the present invention, preferably a thermal acid generator is a compound which, when heated to temperatures greater than 90° C. and less than 250° C., generates an acid. The acid, in combination with the crosslinker, crosslinks the polymer. The antireflective coating layer after heat treatment becomes insoluble in the solvents used for coating photoresists, and furthermore, is also insoluble in the alkaline developer used to image the photoresist. Preferably, the thermal acid generator is activated at 90° C. and more preferably at above 120° C., and even more preferably at above 150° C. The antireflective coating layer is heated for a sufficient length of time to crosslink the coating. Examples of acids and thermal acid generators are butane sulfonic acid, triflic acid, nanoflurobutane sulfonic acid, nitrobenzyl tosylates, such as 2-nitrobenzyl tosylate, 2,4-dinitrobenzyl tosylate, 2,6-dinitrobenzyl tosylate, 4-nitrobenzyl tosylate; benzenesulfonates such as 2-trifluoromethyl-6-nitrobenzyl 4-chlorobenzenesulfonate, 2-trifluoromethyl-6-nitrobenzyl 4-nitro benzenesulfonate; phenolic sulfonate esters such as phenyl, 4-methoxybenzenesulfonate; alkyl ammonium salts of organic acids, such as triethylammonium salt of 10-camphorsulfonic acid, and the like.

Thermal acid generators are preferred over free acids, although free acids may also be used, in the novel antireflective composition, since it is possible that over time the shelf stability of the antireflective solution will be affected by the presence of the acid, if the polymer were to crosslink in solution. Thermal acid generators are only activated when the antireflective film is heated on the substrate. Additionally, mixtures of thermal acids and free acids may be used. Although thermal acid generators are preferred for crosslinking the polymer efficiently, an anti-reflective coating composition comprising the polymer and crosslinking agent may also be used, where heating crosslinks the polymer. Examples of a free acid are, without limitation, strong acids, such as sulfonic acids. Sulfonic acids such as toluene sulfonic acid, triflic acid or mixtures of these are preferred.

Alkyl refers to both straight and branched chain saturated hydrocarbon groups having 1 to 20 carbon atoms, for example, methyl, ethyl, propyl, isopropyl, tertiary butyl, dodecyl, and the like.

Examples of the linear or branched alkylene group can have from 1 to 20 carbon atoms, further 1 to 6 carbon atoms, and include such as, for example, methylene, ethylene, propylene and octylene groups.

Aryl refers to an unsaturated aromatic carbocyclic group of from 6 to 20 carbon atoms having a single ring or multiple condensed (fused) rings and include, but are not limited to, for example, phenyl, tolyl, dimethylphenyl, 2,4,6-trimethylphenyl, naphthyl, anthryl and 9,10-dimethoxyanthryl groups.

Aralkyl refers to an alkyl group containing an aryl group. It is a hydrocarbon group having both aromatic and aliphatic structures, that is, a hydrocarbon group in which an alkyl hydrogen atom is substituted by an aryl group, for example, tolyl, benzyl, phenethyl and naphthylmethyl groups.

Cycloalkyl refers to cyclic alkyl groups of from 3 to 50 carbon atoms having a single cyclic ring or multiple condensed (fused) rings. Examples include cyclopropyl group, cyclopentyl group, cyclohexyl group, cycloheptyl group, cyclooctyl, adamantyl, norbornyl, isoboronyl, camphornyl, dicyclopentyl, .alpha.-pinel, tricyclodecanyl, tetracyclododecyl and androstanyl groups. In these monocyclic or polycyclic cycloalkyl groups, the carbon atom may be substituted by a heteroatom such as oxygen atom.

As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. Illustrative substituents include, for example, those described hereinabove. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The antireflective coating composition can optionally contain a crosslinker.

Examples of crosslinkers include glycoluril-aldehyde resins, melamine-aldehyde resins, benzoguanamine-aldehyde resins, and urea-aldehyde resins. Examples of the aldehyde include formaldehyde, acetaldehyde, etc. In some instances, three or four alkoxy groups are useful. Monomeric, alkylated glycoluril-formaldehyde resins are an example. The glycoluril compounds are known and available commercially, and are further described in U.S. Pat. No. 4,064,191. Glycolurils are synthesized by reacting two moles of urea with one mole of glyoxal. The glycoluril can then be fully or partially methylolated with formaldehyde. One example is tetra(alkoxyalkyl)glycoluril having the following structure

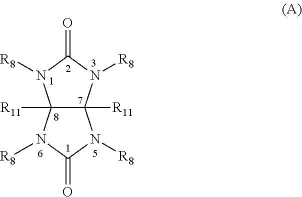

where each R₈ is (CH₂)ₙ—O—W—R₁₂, each R₁₁ is hydrogen or C₁-C₁₀ alkyl, R12 is hydrogen or methyl; W is a direct bond or a straight or branched C₁-C₁₀ alkylene, and n is 0 to 4.
(the numbers in (A) indicating atom number for compound naming)

Examples of tetra(alkoxymethyl)glycoluril, may include, e.g., tetra(methoxymethyl)glycoluril, tetra(ethoxymethyl)glycoluril, tetra(n-propoxymethyl)glycoluril, tetra(i-propoxymethyl)glycoluril, tetra(n-butoxymethyl)glycoluril, tetra(t-butoxymethyl)glycoluril, substituted tetra(alkoxymethyl)glycolurils such as 7-methyl tetra(methoxymethyl)glycoluril, 7-ethyl tetra(methoxymethyl)glycoluril, 7-(i- or n-)propyl tetra(methoxymethyl)glycoluril, 7-(i- or sec- or t-)butyl tetra(methoxymethyl)glycoluril, 7,8-dimethyl tetra(methoxymethyl)glycoluril, 7,8-diethyl tetra(methoxymethyl)glycoluril, 7,8-di(i- or n-)propyl tetra(methoxymethyl)glycoluril, 7,8-di(i- or sec- or t-)butyl tetra(methoxymethyl)glycoluril, 7-methyl-8-(i- or n-)propyl tetra(methoxymethyl)glycoluril, and the like. Tetra(methoxymethyl)glycoluril is available under the trademark POWDERLINK from Cytec Industries (e.g., POWDERLINK 1174). Other examples include methylpropyltetramethoxymethyl glycoluril, and methylphenyltetramethoxymethyl glycoluril.

Other aminoplasts are commercially available from Cytec Industries under the trademark CYMEL and from Monsanto Chemical Co. under the trademark RESIMENE. Condensation products of other amines and amides can also be employed, for example, aldehyde condensates of triazines, diazines, diazoles, guanidines, guanimines and alkyl- and aryl-substituted derivatives of such compounds, including alkyl- and aryl-substituted melamines. Some examples of such compounds are N,N′-dimethyl urea, benzourea, dicyandiamide, formaguanamine, acetoguanamine, ammeline, 2-chloro-4,6-diamino-1,3,5-triazine, 6-methyl-2,4-diamino,1,3,5-traizine, 3,5-diaminotriazole, triaminopyrimidine,2-mercapto-4,6-diamino-pyrimidine, 3,4,6-tris(ethylamino)-1,3,5-triazine, tris(alkoxycarbonylamino)triazine, N,N,N′,N′-tetramethoxymethylurea and the like.

Other possible aminoplasts include compounds having the following structures:

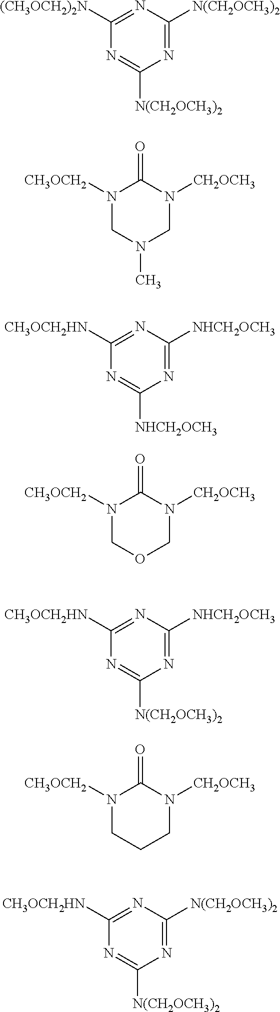

including their analogs and derivatives, such as those found in Japanese Laid-Open Patent Application (Kokai) No. 1-293339 to Tosoh, as well as etherified amino resins, for example methylated or butylated melamine resins (N-methoxymethyl- or N-butoxymethyl-melamine respectively) or methylated/butylated glycolurils, for example as can be found in Canadian Patent No. 1 204 547 to Ciba Specialty Chemicals. Various melamine and urea resins are commercially available under the Nicalacs (Sanwa Chemical Co.), Plastopal (BASF AG), or Maprenal (Clariant GmbH) tradenames.

In some instances, the crosslinker is formed from the condensation reaction of glycoluril with a reactive comonomer containing hydroxy groups and/or acid groups in one case, at least two reactive groups (hydroxy and/or acid) should be available in the comonomer which reacts with the glycoluril. The polymerization reaction may be catalyzed with an acid. In another case, the glycoluril compound may condense with itself or with another polyol, polyacid or hybrid compound, and additionally, incorporate into the polymer a compound with one hydroxy and/or one acid group. Thus the polymer comprises monomeric units derived from glycoluril and reactive compounds containing a mixture of hydroxy and/or acid groups.

The polyhydroxy compound useful as the comonomer for polymerizing with the glycoluril may be a compound containing 2 or more hydroxyl groups or be able to provide 2 or more hydroxyl groups, such as diol, triol, tetrol, glycol, aromatic compounds with 2 or more hydroxyl groups, or polymers with end-capped hydroxyl groups or epoxide groups. More specifically, the polyhydroxy compound may be ethylene glycol, diethylene glycol, propylene glycol, neopentyl glycol, polyethylene glycol, styrene glycol, propylene oxide, ethylene oxide, butylene oxide, hexane diol, butane diol, 1-phenyl-1,2-ethanediol, 2-bromo-2-nitro-1,3-propane diol, 2-methyl-2-nitro-1,3-propanediol, diethylbis(hydroxymethyl)malonate, hydroquinone, and 3,6-dithia-1,8-octanediol. Further examples of aromatic diols are (2,2-bis(4-hydroxyphenyl)propane), 4,4′-isopropylidenebis(2,6-dimethylphenol), bis(4-hydroxyphenyl)methane, 4,4′-sulfonyldephenol, 4,4′-(1,3-phenylenediisopropylidene)bisphenol, 4,4′-(1,4 phenylenediisopropylidene)bisphenol, 4,4′-cyclohexylidenebisphenol, 4,4′-(1-phenylethylidene)bisphenol, 4,4′-ethylidenebisphenol, 2,2-bis(4-hydroxy-3-tert-butylphenyl)propane; 2,2-bis(4-hydroxy-3-methylphenyl)propane, 1,1-bis(4-hydroxyphenyl)ethane; 1,1-bis(4-hydroxyphenyl)isobutane; bis(2-hydroxy-1-naphthyl)methane; 1,5-dihydroxynaphthalene; 1,1-bis(4-hydroxy-3-alkylphenyl)ethane, 2,2-bis(3-sec-butyl-4-hydroxyphenyl)propane, 2,2-bis(4-hydroxy-3-isopropylphenyl)propane, 2,2-bis(4-hydroxyphenyl)butane, α,α′-bis(4-hydroxy-3,5-dimethylphenyl)-1,4-diisopropylbenzene, 2,6-bis(hydroxymethyl)-p-cresol and 2,2′-(1,2-phenylenedioxy)-diethanol, 1,4-benzenedimethanol, 2-benzyloxy-1,3-propanediol, 3-phenoxy-1,2-propanediol, 2,2′-biphenyldimethanol, 4-hydroxybenzyl alcohol, 1,2-benzenedimethanol, 2,2′-(o-phenylenedioxy)diethanol, 1,7-dihydroxynaphthalene, 1,5-naphthalenediol, 9,10-anthracenediol, 9,10-anthracenedimethanol, 2,7,9-anthracenetriol, other naphthyl diols and other anthracyl diols as well as a compound (3) obtained by reacting a compound having the formula

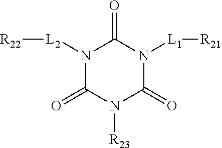

where L₁ and L₂ each independently represent a divalent linking group, R₂₁ and R₂₂ each represent a carbonyl group, and R₂₃ is hydrogen or C₁-C₁₀ alkyl with a polyhydroxy compound,
and mixtures thereof.

Examples of the divalent linking chain include a substituted or unsubstituted alkylene group, substituted or unsubstituted cycloalkylene group, a substituted or unsubstituted arylene group, a substituted or unsubstituted alkylene group having a linking group (such as ether, ester or amido, the same meaning is applied hereinafter) inside the group, and a substituted or unsubstituted arylene group having a linking group inside the group. Examples of the substituent include a halogen atom, a hydroxyl group, a mercapto group, a carboxyl group, an epoxy group, an alkyl group and an aryl group. These substituents may be further substituted with another substituent.

The polyacid compound useful as the reactive comonomer for polymerizing with the glycoluril may be a compound containing 2 or more acid groups or be able to provide 2 or more acidic groups, such as diacid, triacid, tetracid, anhydride, aromatic compounds with 2 or more acid groups, aromatic anhydrides, aromatic dianhydrides, or polymers with end-capped acid or anhydride groups. More specifically, the polyacid compound may be phenylsuccinic acid, benzylmalonic acid, 3-phenylglutaric acid 1,4-phenyldiacetic acid, oxalic acid, malonic acid, succinic acid, pyromellitic dianhydride, 3,3′,4,4′-benzophenone-tetracarboxylic dianhydride, naphthalene dianhydride, 2,3,6,7-naphthalenetetracarboxylic acid dianhydride and 1,4,5,8-naphthalenetetracarboxylic acid dianhydride, and anthracene diacid.

Hybrid compounds containing a mixture of hydroxyl and acid groups may also function as comonomers, and may be exemplified by 3-hydroxyphenylacetic acid and 2-(4-hydroxyphenoxy)propionic acid.

The reaction product between glycoluril and reactive compound is typically done by synthesized by polymerizing the comonomers described previously. Typically, the desired glycoluril or mixtures of glycolurils is reacted with the reactive compound comprising polyol, polyacid, hybrid compound with acid and hydroxyl groups, reactive compound with one hydroxy group, reactive compound with one acid group or mixtures thereof, in the presence of a suitable acid. The polymer may be a linear polymer made with a glycoluril with 2 linking sites that are reacted or a network polymer where the glycoluril has more than 2 reactive sites connected to the polymer. Other comonomers may also be added to the reaction mixture and polymerized to give the polymer of the present invention. Strong acids, such as sulfonic acids are preferred as catalyst for the polymerization reaction. A suitable reaction temperature and time is selected to give a polymer with the desired physical properties, such as molecular weight. Typically the reaction temperature may range from about room temperature to about 150° C. and the reaction time may be from 20 minutes to about 24 hours. The weight average molecular weight (Mw) of the polymer is in the range of 1,000 to 50,000, preferably 3,000 to 40,000, and more preferably 4,500 to 40,000, and even more preferably 5,000 to 35,000 for certain applications. When the weight average molecular weight is low, such as below 1,000, then good film forming properties are not obtained for the antireflective coating and when the weight average molecular weight is too high, then properties such as solubility, storage stability and the like may be compromised. However, lower molecular weight novel polymers of the present invention can function well as crosslinking compounds in conjunction with another crosslinkable polymer, especially where the molecular weight of the lower molecular weight polymer ranges from about 500 to about 20,000, and preferably 800 to 10,000. The reaction product between glycoluril and reactive compound is more fully described in U.S. Ser. No. 11/159,002, the contents of which are hereby incorporated herein by reference.

Examples of compound (3) which are reacted with polyhydroxy compounds include a compound having the formula

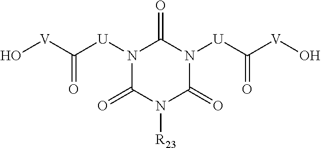

where U is a divalent linking group; V is a direct bond, C₁-C₁₀ straight or branched alkylene, or cycloalkylene group; and R₂₃ is hydrogen or C₁-C₁₀ alkyl. Examples of the divalent linking group include an alkylene group, a phenylene group, a cycloalkylene group, etc.

Examples of the reaction product between compound (3) and polyhydroxy compounds include

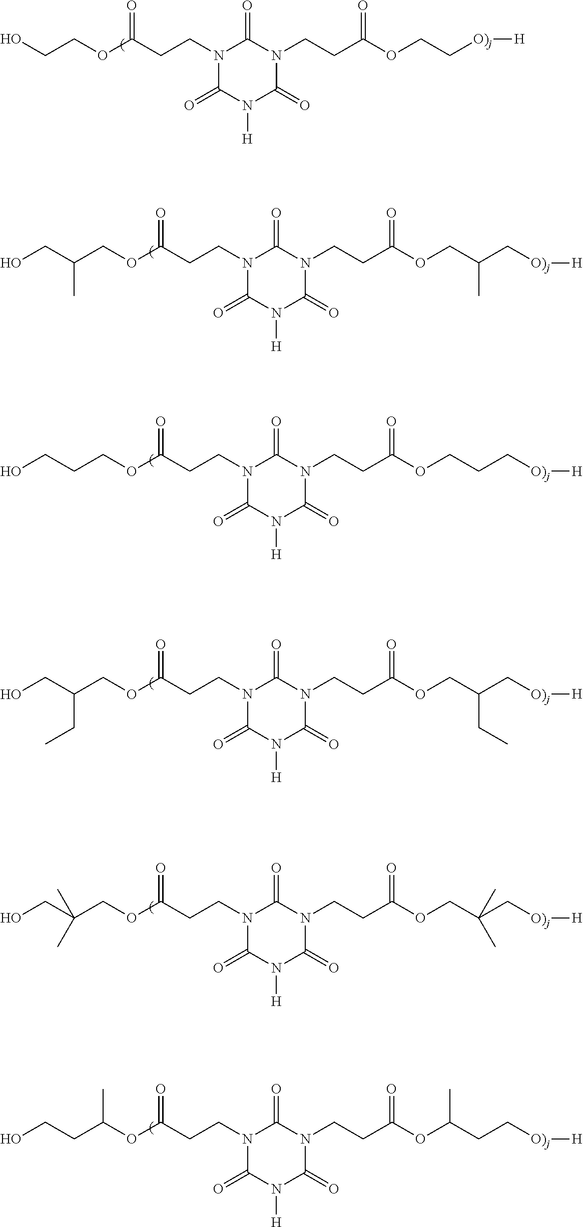

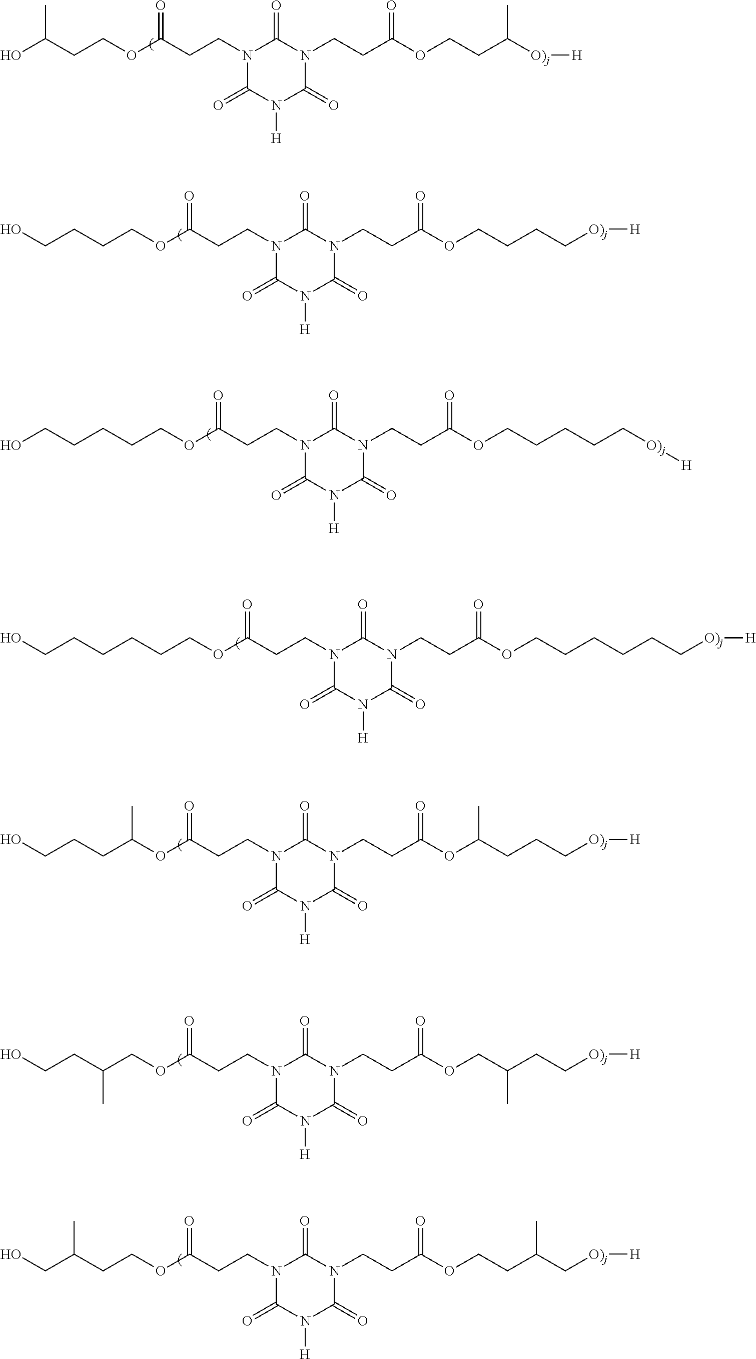

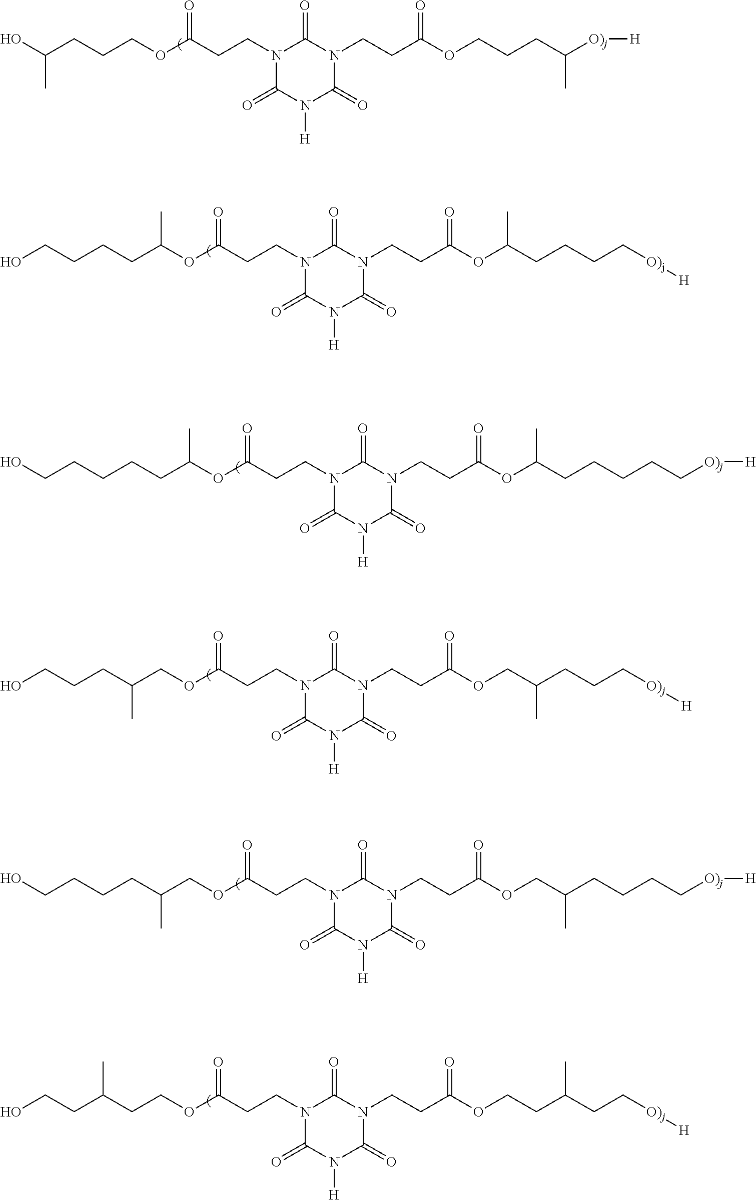

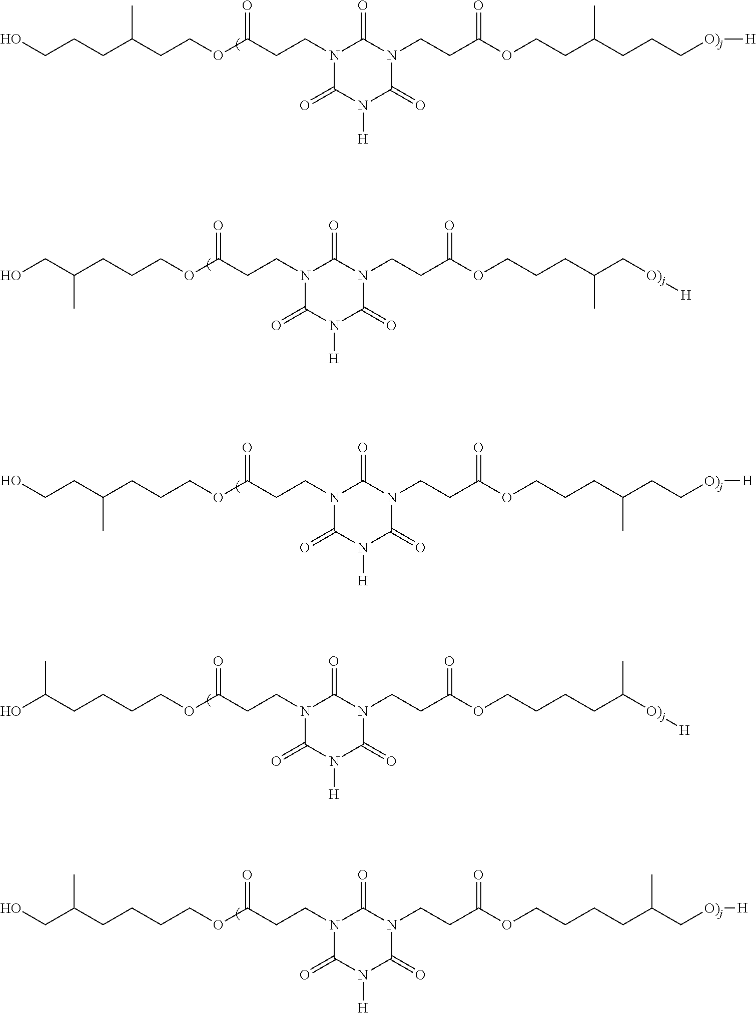

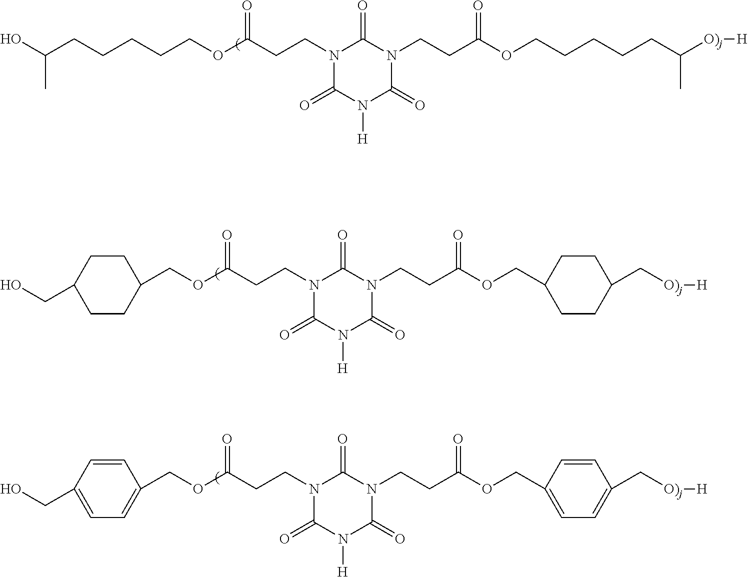

where j is 1 to 5.

The above compounds can be made by reacting the compound (3) with a polyhydroxy compound in the presence of an acid catalyst.

The glycoluril and compound (3) can be reacted together in the presence of or in the absence of another polyhydroxy compound.

One example of the reaction product between glycoluril and compound (3) include a compound having a repeating unit selected from

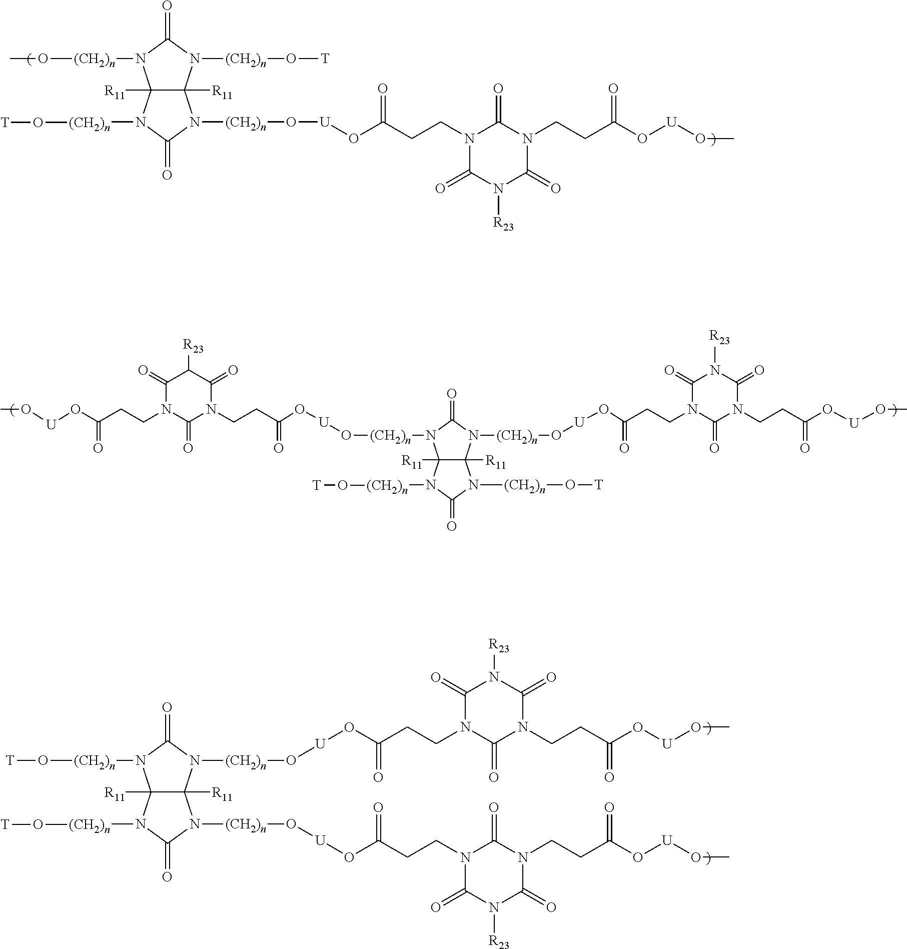

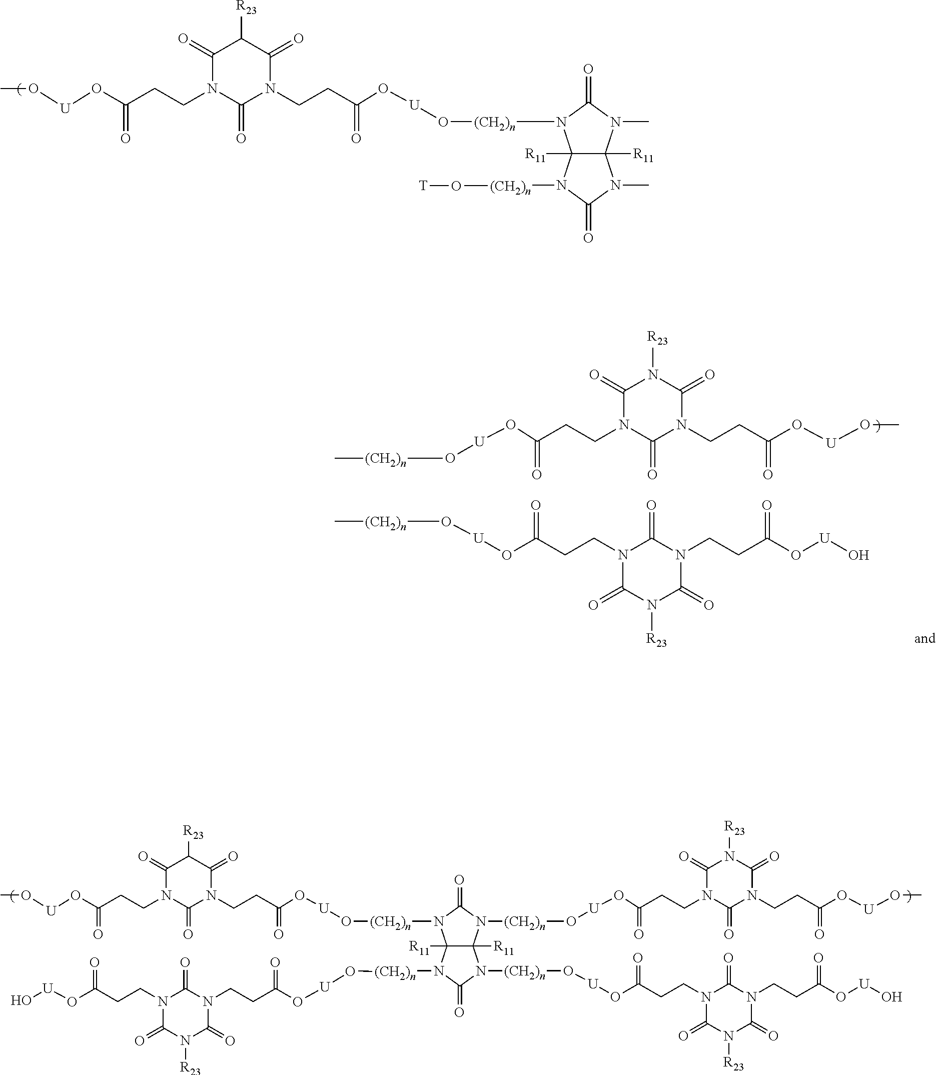

where U is a divalent linking group; V is a direct bond, C₁-C₁₀ straight or branched alkylene, or cycloalkylene group; each R₁₁ is hydrogen or C₁-C₁₀ alkyl; T is hydrogen, a straight or branched C₁-C₁₀ alkyl, or the residue of a polyhydroxy compound; R₂₃ is hydrogen or C₁-C₁₀ alkyl; and n is 0 to 4. Examples of the divalent linking group include an alkylene group, a phenylene group, a cycloalkylene group, etc. Residues of polyhydroxy compound include those from styrene glycol, ethylene glycol, propylene glycol, neopentyl glycol, etc.

One example of the foregoing is

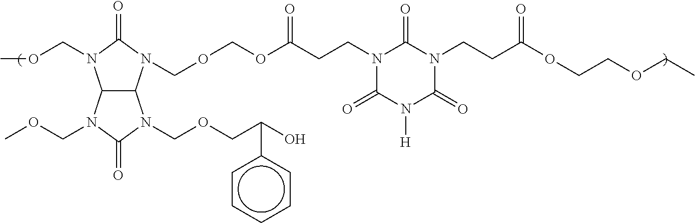

as are

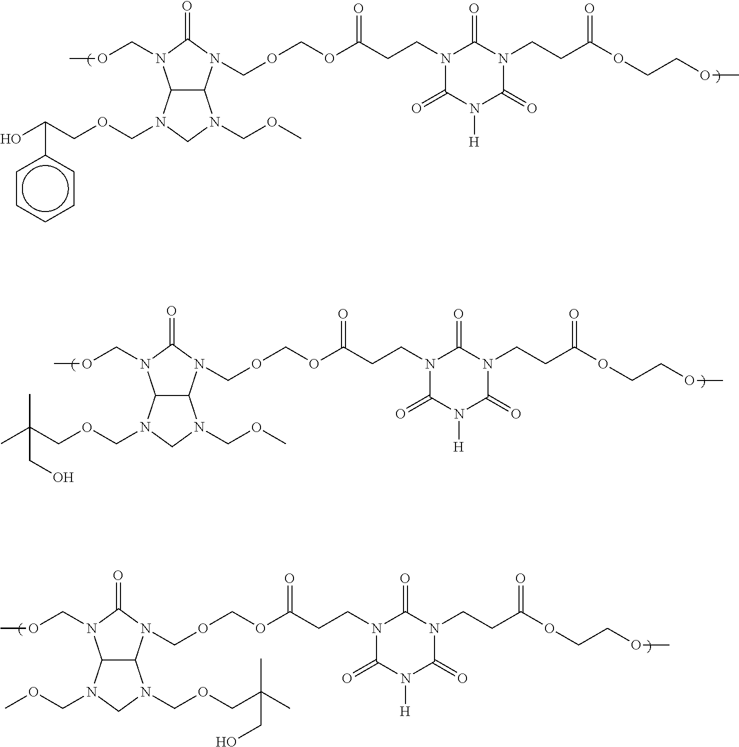

etc, and the like.

The above compounds can be made by the procedures shown in the examples below.

The reactive comonomers, in addition to containing a hydroxyl and/or acid group, may also contain a radiation absorbing chromophore, where the chrompophore absorbs radiation in the range of about 450 nm to about 140 nm. In particular for antireflective coatings useful for imaging in the deep UV (250 nm to 140 nm), aromatic moieties are known to provide the desirable absorption characteristics. These chromophores may be aromatic or heteroaromatic moieties, examples of which are substituted or unsubstituted phenyl, substituted or unsubstituted naphthyl, and substituted or unsubstituted anthracyl. Typically, anthracyl moieties are useful for 248 nm exposure, and phenyl moieties are useful for 193 nm exposure. The aromatic groups may have pendant hydroxy and/or acid groups or groups capable of providing hydroxy or acid groups (e.g. epoxide or anhydride) either attached directly to the aromatic moiety or through other groups, where these hydroxy or acid groups provide the reaction site for the polymerization process. As an example, styrene glycol or an anthracene derivative, may be polymerized with the glycoluril.

Additionally, the chromophore group may be present as an additive, where the additive is a monomeric or polymeric compound. Monomers containing substituted or unsubstituted phenyl, substituted or unsubstituted naphthyl, and substituted or unsubstituted anthracyl may be used. Aromatic polymers function well as chromophoric additives. Example of chromphoric polymers are ones polymerized with at least one or more of the following comonomers: styrene or its derivatives, phenols or its derivatives and an aldehyde, and (meth)acrylates with pendant phenyl, naphthyl or anthracyl groups. More specifically the monomers can be 4-hydroxystyrene, styrene glycol, cresol and formaldehyde, 1-phenyl-1,2-ethanediol, bisphenol A, 2,6-bis(hydroxymethyl)-p-cresol, ethylene glycol phenyl ether acrylate, 2-(4-benzoyl-3-hydroxyphenoxy)ethyl acrylate, 2-hydroxy-3-phenoxypropyl acrylate, benzyl methacrylate, 2,2′-(1,2-phenylenedioxy)-diethanol, 1,4-benzenedimethanol, naphthyl diols, anthracyl diols, phenylsuccinic acid, benzylmalonic acid, 3-phenylglutaric acid, 1,4-phenyldiacetic acid, pyromellitic dianhydride, 3,3′,4,4′-benzophenone-tetracarboxylic dianhydride, naphthalene dianhydride, 2,3,6,7-naphthalenetetracarboxylic acid dianhydride, 1,4,5,8-naphthalenetetracarboxylic acid dianhydride, 9-anthracene methacrylate, and anthracene diacid.

The novel composition may further contain a photoacid generator, examples of which without limitation, are onium salts, sulfonate compounds, nitrobenzyl esters, triazines, etc. The preferred photoacid generators are onium salts and sulfonate esters of hydoxyimides, specifically diphenyl iodnium salts, triphenyl sulfonium salts, dialkyl iodonium salts, triakylsulfonium salts, and mixtures thereof.

Examples of solvents for the coating composition include alcohols, esters, glymes, ethers, glycol ethers, glycol ether esters, ketones, lactones, cyclic ketones, and mixtures thereof. Examples of such solvents include, but are not limited to, propylene glycol methyl ether, propylene glycol methyl ether acetate, cyclohexanone, 2-heptanone, ethyl 3-ethoxy-propionate, propylene glycol methyl ether acetate, ethyl lactate, gamma valerolactone, methyl 3-methoxypropionate, and mixtures thereof. The solvent is typically present in an amount of from about 40 to about 99 weight percent. In certain instances, the addition of lactone solvents is useful in helping flow characteristics of the antireflective coating composition when used in layered systems. When present, the lactone solvent comprises about 1 to about 10% of the solvent system. γ-valerolactone is a useful lactone solvent.

The amount of the compound of (4) in the present composition can vary from about 100 weight % to about 1 weight % relative to the solid portion of the composition. The amount of the crosslinker in the present composition, when used, can vary from 0 weight % to about 50 weight % relative to the solid portion of the composition. The amount of the acid generator in the present composition can vary from 0.1 weight % to about 10 weight % relative to the solid portion of the composition.

The present composition can optionally comprise additional materials typically found in antireflective coating compositions such as, for example, monomeric dyes, lower alcohols, surface leveling agents, adhesion promoters, antifoaming agents, etc, provided that the performance is not negatively impacted.

Since the composition is coated on top of the substrate and is further subjected to dry etching, it is envisioned that the composition is of sufficiently low metal ion level and purity that the properties of the semiconductor device are not adversely affected. Treatments such as passing a solution of the polymer, or compositions containing such polymers, through an ion exchange column, filtration, and extraction processes can be used to reduce the concentration of metal ions and to reduce particles.

The optical characteristics of the antireflective coating are optimized for the exposure wavelength and other desired lithographic characteristics. As an example the absorption parameter (k) of the novel composition for 193 nm exposure ranges from about 0.1 to about 1.0, preferably from about 0.1 to about 0.75, more preferably from about 0.1 to about 0.35 as measured using ellipsometry. The value of the refractive index (n) ranges from about 1.25 to about 2.0, preferably from about 1.8 to about 2.0. Due to the good absorption characteristics of this composition at 193 nm, very thin antireflective films of the order of about 20 nm may be used. This is particularly advantageous when using a nonaromatic photoresist, such as those sensitive at 193 nm, 157 nm and lower wavelengths, where the photoresist films are thin and must act as an etch mask for the antireflective film.

The substrates over which the antireflective coatings are formed can be any of those typically used in the semiconductor industry. Suitable substrates include, without limitation, silicon, silicon substrate coated with a metal surface, copper coated silicon wafer, copper, substrate coated with antireflective coating, aluminum, polymeric resins, silicon dioxide, metals, doped silicon dioxide, silicon nitride, silicon oxide nitride, titanium nitride, tantalum, tungsten, copper, polysilicon, ceramics, aluminum/copper mixtures; gallium arsenide and other such Group III/V compounds, and the like. The substrate may comprise any number of layers made from the materials described above.

The coating composition can be coated on the substrate using techniques well known to those skilled in the art, such as dipping, spincoating or spraying. The film thickness of the anti-reflective coating ranges from about 0.01 μm to about 1 μm. The coating can be heated on a hot plate or convection oven or other well known heating methods to remove any residual solvent and induce crosslinking if desired, and insolubilizing the anti-reflective coatings to prevent intermixing between the anti-reflective coating and the photoresist. The preferred range of temperature is from about 90° C. to about 250° C. If the temperature is below 90° C. then insufficient loss of solvent or insufficient amount of crosslinking takes place, and at temperatures above 300° C. the composition may become chemically unstable. A film of photoresist is then coated on top of the uppermost antireflective coating and baked to substantially remove the photoresist solvent. An edge bead remover may be applied after the coating steps to clean the edges of the substrate using processes well known in the art.

There are two types of photoresist compositions, negative-working and positive-working. When negative-working photoresist compositions are exposed image-wise to radiation, the areas of the resist composition exposed to the radiation become less soluble to a developer solution (e.g. a cross-linking reaction occurs) while the unexposed areas of the photoresist coating remain relatively soluble to such a solution. Thus, treatment of an exposed negative-working resist with a developer causes removal of the non-exposed areas of the photoresist coating and the creation of a negative image in the coating, thereby uncovering a desired portion of the underlying substrate surface on which the photoresist composition was deposited.

On the other hand, when positive-working photoresist compositions are exposed image-wise to radiation, those areas of the photoresist composition exposed to the radiation become more soluble to the developer solution (e.g. a rearrangement reaction occurs) while those areas not exposed remain relatively insoluble to the developer solution. Thus, treatment of an exposed positive-working photoresist with the developer causes removal of the exposed areas of the coating and the creation of a positive image in the photoresist coating. Again, a desired portion of the underlying surface is uncovered.

Negative working photoresist and positive working photoresist compositions and their use are well known to those skilled in the art.

A process of the instant invention comprises coating a substrate with an antireflective coating composition comprising a polymer of the present invention and heating the substrate on a hotplate or convection oven or other well known heating methods at a sufficient temperature for sufficient length of time to remove the coating solvent, and crosslink the polymer if necessary, to a sufficient extent so that the coating is not soluble in the coating solution of a photoresist or in a aqueous alkaline developer. An edge bead remover may be applied to clean the edges of the substrate using processes well known in the art. The heating ranges in temperature from about 70° C. to about 250° C. If the temperature is below 70° C., then insufficient loss of solvent or insufficient amount of crosslinking may take place, and at temperatures above 250° C., the polymer may become chemically unstable. A film of a photoresist composition is then coated on top of the antireflective coating and baked to substantially remove the photoresist solvent. The photoresist is image-wise exposed and developed in an aqueous developer to remove the treated resist. An optional heating step can be incorporated into the process prior to development and after exposure. The process of coating and imaging photoresists is well known to those skilled in the art and is optimized for the specific type of resist used. The patterned substrate can then be dry etched in a suitable etch chamber to remove the exposed portions of the anti-reflective film, with the remaining photoresist acting as an etch mask. Various gases are known in the art for etching organic antireflective coatings, such as O₂, Cl₂, F₂ and CF₄ as well as other etching gases known in the art. This process is generally known as a bilayer process.

An intermediate layer may be placed between the antireflective coating and the photoresist to prevent intermixing, and is envisioned as lying within the scope of this invention. The intermediate layer is an inert polymer cast from a solvent, where examples of the polymer are polysulfones and polyimides.

In addition, a multilayer system, for example, a trilayer system, or process is also envisioned within the scope of the invention. In a trilayer process for example, an organic film is formed on a substrate, an antireflection film is formed on the organic film, and a photoresist film is formed on the antireflection film. The organic film can also act as an antireflection film. The organic film is formed on a substrate as a lower resist film by spin coating method etc. The organic film may or may not then crosslinked with heat or acid after application by spin coating method etc. On the organic film is formed the antireflection film, for example that which is disclosed herein, as an intermediate resist film. After applying the antireflection film composition to the organic film by spin-coating etc., an organic solvent is evaporated, and baking is carried out in order to promote crosslinking reaction to prevent the antireflection film from intermixing with an overlying photoresist film. After the antireflection film is formed, the photoresist film is formed thereon as an upper resist film. Spin coating method can be used for forming the photoresist film as with forming the antireflection film. After photoresist film composition is applied by spin-coating method etc., pre-baking is carried out. After that, a pattern circuit area is exposed, and post exposure baking (PEB) and development with a developer are carried out to obtain a resist pattern.

Another trilayer resist process is such when a bottom layer is formed with a carbon etch mask. On top of the bottom layer, an intermediate layer is formed by using an intermediate resist layer composition containing silicon atoms. On top of the intermediate layer, an antireflection layer based on the antireflection coating composition of the present invention, is formed. Finally, on top of the antireflection layer, a top layer is formed by using a top resist layer composition of a photoresist composition. In this case, examples of the composition for forming the intermediate layer may include polysilsesquioxane-based silicone polymer, tetraorthosilicate glass (TEOS), and the like. Then films prepared by spin-coating such a composition, or a film of SiO₂, SiN, or SiON prepared by CVD may be used as the intermediate layer. The top resist layer composition of a photoresist composition preferably comprises a polymer without a silicon atom. A top resist layer comprising a polymer without a silicon atom has an advantage of providing superior resolution to a top resist layer comprising a polymer containing silicon atoms. Then in the same fashion as the bilayer resist process mentioned above, a pattern circuit area of the top resist layer is exposed according to standard procedures. Subsequently, post exposure baking (PEB) and development are carried out to obtain a resist pattern, followed by etching and further lithographic processes.

The following examples provide detailed illustrations of the methods of producing and utilizing compositions of the present invention. These examples are not intended, however, to limit or restrict the scope of the invention in any way and should not be construed as providing conditions, parameters or values which must be utilized exclusively in order to practice the present invention.

### SYNTHESIS EXAMPLES

### Synthetic Example 1

66 g of propylene glycol monomethyl ether, 4.098 g (0.015 mol) of bis(2-carboxyethyl)isocyanurate, 1.80 g (0.015 mol) of styrene oxide and 0.05 g (2.2×10⁴ mol) of benzyltriethylammonium chloride were charged into a suitably sized flask having a thermometer, a cold water condenser, a mechanical stirrer, an external heating source, and nitrogen source. Under nitrogen, the materials were dissolved with stirring and the temperature was raised to 110° C. and maintained at this temperature for 24 hours. At the end of 24 hours, the reaction solution was cooled down to 90° C., and then 1.49 g (0.005 mol) of tris(2,3-epoxypropyl)isocyanurate was added and the reaction mixture was kept at 90° C. for 3 hrs and then raised to 100° C. for 3 hrs. The reaction mixture was then cooled down to room temperature and used as is. The GPC analysis of the resulting polymer showed that it had a number average molecular weight Mn of 2678 and a weight average molecular weight Mw of 4193 (in terms of standard polystyrene).

### Synthetic Example 2

177 g of propylene glycol monomethyl ether, 13.66 g (0.05 mol) of bis(2-carboxyethyl)isocyanurate, 12.0 g (0.10 mol) of styrene oxide and 0.10 g (4.4×10⁻⁴ mol) of benzyltriethylammonium chloride were charged into a suitably sized flask having a thermometer, a cold water condenser, a mechanical stirrer, an external heating source, and nitrogen source. Under nitrogen, the materials were dissolved with stirring and the temperature was raised to 120° C. After Kept the reaction reflux for 24 hours, the reaction solution was cooled down to 90° C., and then 4.95 g (0.0167 mol) of tris(2,3-epoxypropyl)isocyanurate was added and the reaction mixture was kept at the reflux temperature for 7 hrs. The reaction mixture was then cooled down to room temperature and used as is. GPC analysis of the resulting polymer showed that it had a number average molecular weight Mn of 2547 and a weight average molecular weight Mw of 5106 (in terms of standard polystyrene).

### Synthetic Example 3

150 g of propylene glycol monomethyl ether, 27.32 g (0.1 mol) of bis(2-carboxyethyl)isocyanurate, 9.25 g (0.10 mol) of epichlorohydrin and 0.10 g (4.4×10⁻⁴ mol) of benzyltriethylammonium chloride were charged into a suitably sized flask having a thermometer, a cold water condenser, a mechanical stirrer, an external heating source, and nitrogen source. Under nitrogen, the materials were dissolved with stirring and the temperature was raised to 120° C. and maintained at this temperature for 24 hours. At the end of 24 hours, 12.0 g (0.10 mol) of styrene oxide was added. The reaction was then continued at reflux temperature for another 24 hours. Thereafter, 9.91 g (0.033 mol) of tris(2,3-epoxypropyl)isocyanurate was added to the mixture and the reaction mixture was kept at the reflux temperature for another 24 hrs. The reaction mixture was then cooled down to room temperature and used as is. GPC analysis of the resulting polymer showed that it had a number average molecular weight Mn of 4588 and a weight average molecular weight Mw of 7193 (in terms of standard polystyrene).

### Synthetic Example 4

149 g of propylene glycol monomethyl ether, 16.39 g (0.06 mol) of bis(2-carboxyethyl)isocyanurate, 9.85 g (0.06 mol) of benzyl glycidyl ether and 0.15 g (6.6×10⁻⁴ mol) of benzyltriethylammonium chloride were charged into a suitably sized flask having a thermometer, a cold water condenser, a mechanical stirrer, an external heating source, and nitrogen source. Under nitrogen, the materials were dissolved with stirring and the temperature was raised to reflux temperature (about 118° C.). After stirring under nitrogen atmosphere at the reflux temperature for 24 hours, the reaction solution was cooled down to 90° C., and 5.95 g (0.02 mol) of tris(2,3-epoxypropyl)isocyanurate was added. The reaction mixture was kept at 90° C. for 16 hrs. The reaction mixture was then cooled down to room temperature and used as is. GPC analysis of the resulting polymer showed that it had a number average molecular weight Mn of 4077 and a weight average molecular weight Mw of 6149 (in terms of standard polystyrene).

### Synthetic Example 5

Into a suitably sized flask having a thermometer, a Dean-Stark trap, a mechanical stirrer, an external heating source, and nitrogen source were placed 27.3 g (0.10 mol) of bis(2-carboxyethyl)isocyanurate, 12.4 g (0.20 mol) of ethylene glycol 0.25 g (1.31×10⁻³ mol) of para-toluenesulfonic acid monohydrate. The temperature of the mixture was raised to 140° C. and was maintained at this temperature with stirring under nitrogen until the evolution of water ceased. The reaction solution was cooled down to 90° C. and 191 g of acetonitrile was added to dissolve the reaction product, and then with some further cooling, 21.2 g (0.0667 mol) of tetramethoxy methyl glycoluril was added at 80° C. The reaction mixture was kept at 80° C. for 6 hrs. The reaction was terminated by adding 0.25 g of triethylamine to the reaction mixture. The reaction mixture was cooled down to room temperature and then precipitated in DI-water. The solid polymer was washed and dried under vacuum at 40° C., yielding 35.0 g (69%). GPC analysis of the resulting polymer showed that it had a number average molecular weight Mn of 5006 and a weight average molecular weight Mw of 8135 (in terms of standard polystyrene).

### Synthetic Example 6

600 grams of tetramethoxymethyl glycoluril, 96 grams of styrene glycol and 1200 grams of propylene glycol monomethyl ether acetate (PGMEA) were charged into a 2 liter(l) jacketed flask fitted with a thermometer, mechanical stirrer, nitrogen source, and a cold water condenser and heated to 85° C. A catalytical amount of para-toluenesulfonic acid monohydrate was added, and the reaction was maintained at this temperature for 5 hrs. The reaction solution was then cooled to room temperature and filtered. The filtrate was slowly poured into distilled water to precipitate the polymer. The polymer was filtered, washed thoroughly with water and dried in a vacuum oven (250 grams of the polymer were obtained). The polymer obtained had a weight average molecular weight of about 17,345 g/mol and a polydispersity of 2.7. H¹NMR showed that the polymer was a condensation product of the two starting materials. A broad peak centered at 7.3 ppm was indicative of the benzene moiety present in the polymer and the broad peak centered at 3.3 ppm was contributed by unreacted methoxy groups (CH₃O) on tetramethoxymethyl glycoluril.

### Synthetic Example 7

260 grams of tetramethoxymethyl glycoluril, 41.6 grams of neopentyl glycol and 520 grams of PGMEA were charged into a 2 l jacketed flask fitted with a thermometer, mechanical stirrer, nitrogen source, and a cold water condenser and heated to 85° C. A catalytical amount of para-toluenesulfonic acid monohydrate was added, and the reaction was maintained at this temperature for 5 hrs. The reaction solution was then cooled to room temperature and filtered. The filtrate was slowly poured into distilled water while stirring in order to precipitate the polymer. The polymer was filtered, washed thoroughly with water and dried in a vacuum oven (250 grams of the polymer were obtained). The polymer obtained had a weight average molecular weight of about 18,300 g/mol and a polydispersity of 2.8. A broad peak centered at 0.9 ppm was assigned to methyl groups of neopentyl glycol and the broad peak centered at 3.3 ppm is characteristic of unreacted methoxy groups (CH₃O) on tetramethoxymethyl glycoluril, showing that the polymer obtained was a condensation product of the two starting materials.

### Synthetic Example 8

To a 2-Liter flask equipped with a mechanical stirrer, a heating mantle, nitrogen source, and a temperature controller were added 400 grams of MX270 (a glycoluril available from Sanwa Chemicals, Japan), 132 grams of neopentyl glycol and 1050 grams of PGMEA. The solution was stirred at 85° C. When the reaction temperature reached 85° C., 6.0 grams of para-toluenesulfonic acid monohydrate was added. The reaction mixture was kept at 85° C. for 6 hours. The heater was turned off and 3.2 grams of triethylamine added. When the reaction mixture cooled down to room temperature, a white gum polymer was isolated. The polymer was transferred to a container and dried under the vacuum to give a white brittle polymer. The polymer product was analyzed by GPC and had a molecular weight ranging from 800 to 10,000, and with a weight average molecular weight of about 5,000.

### Synthetic Example 9

Into a suitably sized flask having a thermometer, a Dean-Stark trap, a mechanical stirrer, an external heating source, and nitrogen source were placed 27.3 g (0.10 mol) of bis(2-carboxyethyl)isocyanurate, 12.4 g (0.20 mol) of ethylene glycol 0.25 g (1.31×10⁻³ mol) of para-toluenesulfonic acid monohydrate. The temperature of the mixture was raised to 140° C. and was maintained at this temperature with stirring under nitrogen until the evolution of water ceased. The reaction solution was cooled down to 90° C. and 110 g of cyclohexanone was added to dissolve the reaction product, and then with some further cooling, 8.29 g (0.06 mol) of styrene glycol and 50.88 g (0.16 mol) of tetramethoxy methyl glycoluril was added at 80° C. The reaction mixture was kept at 80° C. for 9 hrs. The reaction was terminated by adding 0.25 g of triethylamine to the reaction mixture. The reaction mixture was cooled down to room temperature and then precipitated in 2-propanol. The solid polymer was washed and dried under vacuum at 40° C., yielding 34.0 g (40%). GPC analysis of the resulting polymer showed that it had a number average molecular weight Mn of 4083 and a weight average molecular weight Mw of 6091 (in terms of standard polystyrene).

### Formulation Example 1

40.0 g of the polymer solution obtained in Synthetic Example 1 containing 4.0 g of polymer, and 0.04 g of dodecylbenzenesulfonic acid/triethylamine salt were dissolved in 60.0 g of ethyl lactate to obtain a solution. Then the solution was filtered through a micro filter made of polyethylene having a pore diameter of 0.05 μm, to prepare a composition solution for forming a bottom anti-reflective coating. Refractive index (n) and absorption parameter (k) at a wavelength of 193 nm were measured by spectroscopic ellipsometry. The refractive index (n) was 2.00 and absorption parameter (k) was 0.47.

### Formulation Example 2

40.0 g of the polymer solution obtained in Synthetic Example 2 containing 4.0 g of polymer, and 0.04 g of dodecylbenzenesulfonic acid/triethylamine salt were dissolved in 60.0 g of ethyl lactate to obtain a solution. Then the solution was filtered through a micro filter made of polyethylene having a pore diameter of 0.05 μm, to prepare a composition solution for forming bottom anti-reflective coating. Refractive index (n) and absorption parameter (k) at a wavelength of 193 nm were measured by spectroscopic ellipsometry. The refractive index (n) was 2.03 and absorption parameter (k) was 0.53.

### Formulation Example 3

40.0 g of the polymer solution obtained in Synthetic Example 3 containing 4.0 g of polymer, and 0.04 g of dodecylbenzenesulfonic acid/triethylamine salt were dissolved in 6.0 g of ethyl lactate to obtain a solution. Then the solution was filtered through a micro filter made of polyethylene having a pore diameter of 0.05 μm, to prepare a composition solution for forming bottom anti-reflective coating. Refractive index (n) and absorption parameter (k) at a wavelength of 193 nm were measured by spectroscopic ellipsometry. The refractive index (n) was 1.95 and absorption parameter (k) was 0.37.

### Formulation Example 4

40.0 g of the polymer solution obtained in Synthetic Example 4 containing 4.0 g of polymer, and 0.04 g of dodecylbenzenesulfonic acid/triethylamine salt were dissolved in 60.0 g of ethyl lactate to obtain a solution. Then the solution was filtered through a micro filter made of polyethylene having a pore diameter of 0.05 μm to prepare a composition solution for forming bottom anti-reflective coating. Refractive index (n) and absorption parameter (k) at a wavelength of 193 nm were measured with a spectroscopic ellipsometer. The refractive index (n) was 1.94 and absorption parameter (k) was 0.44.

### Formulation Example 5

35 g of the polymer solution obtained in Synthetic Example 1 containing 3.5 g of polymer, 1.5 g of material from Synthesis Example 7 and 0.045 g of dodecylbenzenesulfonic acid/triethylamine salt were dissolved in 65.0 g of ethyl lactate to obtain a solution. Then the solution was filtered through a micro filter made of polyethylene having a pore diameter of 0.05 μm to prepare a composition solution for forming bottom anti-reflective coating. Refractive index (n) and absorption parameter (k) at a wavelength of 193 nm were measured with a spectroscopic ellipsometer. The refractive index (n) was 1.98 and absorption parameter (k) was 0.40.

### Formulation Example 6

35 g of the polymer solution obtained in Synthetic Example 1 containing 3.5 g of polymer, 1.5 g of product from Synthesis Example 6 and 0.045 g of dodecylbenzenesulfonic acid/triethylamine salt were dissolved in 63.45 g of ethyl lactate to obtain a solution. Then the solution was filtered through a micro filter made of polyethylene having a pore diameter of 0.05 μm to prepare a composition solution for forming bottom anti-reflective coating. Refractive index (n) and absorption parameter (k) at a wavelength of 193 nm were measured with a spectroscopic ellipsometer. The refractive index (n) was 1.99 and absorption parameter (k) was 0.44.

### Formulation Example 7

35 g of the polymer solution obtained in Synthetic Example 1 containing 3.5 g of polymer, 1.5 g of product from Synthesis Example 8 and 0.045 g of dodecylbenzenesulfonic acid/triethylamine salt were dissolved in 63.45 g of ethyl lactate to obtain a solution. Then the solution was filtered through a micro filter made of polyethylene having a pore diameter of 0.05 μm to prepare a composition solution for forming bottom anti-reflective coating. Refractive index (n) and absorption parameter (k) at a wavelength of 193 nm were measured with a spectroscopic ellipsometer. The refractive index (n) was 1.97 and absorption parameter (k) was 0.40.

### Formulation Example 8

30 g of the polymer solution obtained in Synthetic Example 1 containing 3.0 g of polymer, 1.5 g of product from Synthetic Example 5, and 0.045 g of dodecylbenzenesulfonic acid/triethylamine salt were dissolved in 68.45 g of ethyl lactate to obtain a solution. Then the solution was filtered through a micro filter made of polyethylene having a pore diameter of 0.05 μm to prepare a composition solution for forming bottom anti-reflective coating. Refractive index (n) and absorption parameter (k) at a wavelength of 193 nm were measured with a spectroscopic ellipsometer. The refractive index (n) was 2.00 and absorption parameter (k) was 0.40.

### Formulation Example 9

22.5 g of the polymer solution obtained in Synthetic Example 1 containing 2.25 g of polymer, 2.25 g of the material from Synthetic Example 5 and 0.045 g of dodecylbenzenesulfonic acid/triethylamine salt were dissolved in 75.2 g of ethyl lactate to obtain a solution. Then the solution was filtered through a micro filter made of polyethylene having a pore diameter of 0.05 μm to prepare a composition solution for forming bottom anti-reflective coating. Refractive index (n) and absorption parameter (k) at a wavelength of 193 nm were measured with a spectroscopic ellipsometer. The refractive index (n) was 1.99 and absorption parameter (k) was 0.35.

### Formulation Example 10

15.0 9 of the polymer solution obtained in Synthetic Example 1 containing 1.5 g of polymer, 3.0 g of the material from Synthetic Example 5 and 0.045 g of dodecylbenzenesulfonic acid/triethylamine salt were dissolved in 82.0 g of ethyl lactate to obtain a solution. Then the solution was filtered through a micro filter made of polyethylene having a pore diameter of 0.05 μm to prepare a composition solution for forming bottom anti-reflective coating. Refractive index (n) and absorption parameter (k) at a wavelength of 193 nm were measured with a spectroscopic ellipsometer. The refractive index (n) was 1.97 and absorption parameter (k) was 0.30.

### Formulation Example 11

4.5 g of the material from Synthetic Example 5 and 0.045 g of dodecylbenzenesulfonic acid/triethylamine salt were dissolved in 95.45 g of ethyl lactate to obtain a solution. Then the solution was filtered through a micro filter made of polyethylene having a pore diameter of 0.05 μm to prepare a composition solution for forming bottom anti-reflective coating. Refractive index (n) and absorption parameter (k) at a wavelength of 193 nm were measured with a spectroscopic ellipsometer. The refractive index (n) was 1.95 and absorption parameter (k) was 0.21.

### Formulation Example 12

4.5 g of the material from Synthetic Example 9 and 0.045 g of dodecylbenzenesulfonic acid/triethylamine salt were dissolved in 95.45 g of ethyl lactate to obtain a solution. Then the solution was filtered through a micro filter made of polyethylene having a pore diameter of 0.05 μm to prepare a composition solution for forming bottom anti-reflective coating. Refractive index (n) and absorption parameter (k) at a wavelength of 193 nm were measured with a spectroscopic ellipsometer. The refractive index (n) was 1.95 and absorption parameter (k) was 0.22.

### Lithography Example 1

A silicon substrate coated with a bottom antireflective coating (B.A.R.C.) was prepared by spin coating the bottom anti-reflective coating solution of Formulation Example 5 onto the silicon substrate and baking at 220° C. for 60 sec. The optimum B.A.R.C film thickness was 73 nm, which was simulated and determined using PROLITH (v.9.3.5). AZ photoresist (T85531; available from AZ Electronic Materials USA Corp.) was then coated on the B.A.R.C coated silicon substrate. The spin speed was adjusted such that the photoresist film thickness was 150 nm. The coated wafer was then soft baked at 100° C./60 sec, exposed with Nikon 306D 0.85NA & 0.82/0.55 Dipole-Y Illumination using attenuated phase shift mask, post exposure baked at 110° C./160 sec, and developed using a 2.38 weight % aqueous solution of tetramethyl ammonium hydroxide for 30 sec. 75 nm and 80 nm 1:1 line and space patterns were then observed on a scanning electron microscope. The photoresist had very good exposure latitude, good LER and profile shape. The line and space patterns at 75 nm and 80 nm 1:1 duty ratio showed no standing waves, no footing/scum and good collapse margin, indicating the good lithographic performance of the bottom anti-reflective coating.

### Lithography Example 2

A silicon substrate coated with a bottom antireflective coating (B.A.R.C.) was prepared by spin coating the bottom anti-reflective coating solution of Formulation Example 5 onto the silicon substrate and baking at 220° C. for 60 sec. The optimum B.A.R.C film thickness was 28 nm which was simulated and determined using PROLITH (v.9.3.5). A model immersion photoresist was then coated on the B.A.R.C coated silicon substrate. The spin speed was adjusted such that the photoresist film thickness was 110 nm. The coated wafer was then soft baked at 95° C./60 sec, exposed with ASML 1700i 1.20NA & 0.979/0.824 Dipole-40Y Illumination using attenuated phase shift mask, post exposure baked at 90° C./60 sec, and developed using a 2.38 weight % aqueous solution of tetramethyl ammonium hydroxide for 10 sec. 45 nm 1:1 line and space patterns were then observed on a scanning electron microscope. The photoresist had very good exposure latitude, good LER and profile shape. The line and space patterns at 45 nm 1:1 duty ratio showed no standing waves, no footing/scum and good collapse margin indicating the good lithographic performance of the bottom anti-reflective coating.

### Lithography Example 3

A silicon substrate coated with a bottom antireflective coating (B.A.R.C.) was prepared by spin coating the bottom anti-reflective coating solution of Formulation Example 6 onto the silicon substrate and baking at 220° C. for 60 sec. The optimum B.A.R.C film thickness was 73 nm, which was simulated and determined using PROLITH (v.9.3.5). AZ photoresist (T85531; available from AZ Electronic Materials USA Corp.) was then coated on the B.A.R.C coated silicon substrate. The spin speed was adjusted such that the photoresist film thickness was 150 nm. The coated wafer was then soft baked at 100° C./60 sec, exposed with Nikon 306D 0.85NA & 0.82/0.55 Dipole-Y Illumination using attenuated phase shift mask, post exposure baked at 110° C./60 sec, and developed using a 2.38 weight % aqueous solution of tetramethyl ammonium hydroxide for 30 sec. 75 nm and 80 nm 1:1 line and space patterns were then observed on a scanning electron microscope. The photoresist had very good exposure latitude, good LER and profile shape. The line and space patterns at 75 nm and 80 nm 1:1 duty ratio showed no standing waves, no footing/scum and good collapse margin, indicating the good lithographic performance of the bottom anti-reflective coating.

### Lithography Example 4

A silicon substrate coated with a bottom antireflective coating (B.A.R.C.) was prepared by spin coating the bottom anti-reflective coating solution of Formulation Example 8 onto the silicon substrate and baking at 220° C. for 60 sec. The optimum B.A.R.C film thickness was 72 nm, which was simulated and determined using PROLITH (v.9.3.5). AZ photoresist (T85531; available from AZ Electronic Materials USA Corp.) was then coated on the B.A.R.C coated silicon substrate. The spin speed was adjusted such that the photoresist film thickness was 150 nm. The coated wafer was then soft baked at 100° C./60 sec, exposed with Nikon 306D 0.85NA & 0.82/0.55 Dipole-Y Illumination using attenuated phase shift mask, post exposure baked at 110° C./160 sec, and developed using a 2.38 weight % aqueous solution of tetramethyl ammonium hydroxide for 30 sec. 75 nm and 80 nm 1:1 line and space patterns were then observed on a scanning electron microscope. The photoresist had very good exposure latitude, good LER and profile shape. The line and space patterns at 75 nm and 80 nm 1:1 duty ratio showed no standing waves, no footing/scum and good collapse margin, indicating the good lithographic performance of the bottom anti-reflective coating.

### Lithography Example 5

A silicon substrate coated with a bottom antireflective coating (B.A.R.C.) was prepared by spin coating the bottom anti-reflective coating solution of Formulation Example 9 onto the silicon substrate and baking at 220° C. for 60 sec. The optimum B.A.R.C film thickness was 73 nm, which was simulated and determined using PROLITH (v.9.3.5). AZ photoresist (T85531; available from AZ Electronic Materials USA Corp.) was then coated on the B.A.R.C coated silicon substrate. The spin speed was adjusted such that the photoresist film thickness was 150 nm. The coated wafer was then soft baked at 100° C./60 sec, exposed with Nikon 306D 0.85NA & 0.82/0.55 Dipole-Y Illumination using attenuated phase shift mask, post exposure baked at 110° C./60 sec, and developed using a 2.38 weight % aqueous solution of tetramethyl ammonium hydroxide for 30 sec. 75 nm and 80 nm 1:1 line and space patterns were then observed on a scanning electron microscope. The photoresist had very good exposure latitude, good LER and profile shape. The line and space patterns at 75 nm and 80 nm 1:1 duty ratio showed no standing waves, no footing/scum and good collapse margin, indicating the good lithographic performance of the bottom anti-reflective coating.

### Lithography Example 6

A silicon substrate coated with a bottom antireflective coating (B.A.R.C.) was prepared by spin coating the bottom anti-reflective coating solution of Formulation Example 11 onto the silicon substrate and baking at 220° C. for 60 sec. The optimum B.A.R.C film thickness was 78 nm, which was simulated and determined using PROLITH (v.9.3.5). AZ photoresist (T85531; available from AZ Electronic Materials USA Corp.) was then coated on the B.A.R.C coated silicon substrate. The spin speed was adjusted such that the photoresist film thickness was 150 nm. The coated wafer was then soft baked at 100° C./60 sec, exposed with Nikon 306D 0.85NA & 0.82/0.55 Dipole-Y Illumination using attenuated phase shift mask, post exposure baked at 110° C./60 sec, and developed using a 2.38 weight % aqueous solution of tetramethyl ammonium hydroxide for 30 sec. 75 nm and 80 nm 1:1 line and space patterns were then observed on a scanning electron microscope. The photoresist had very good exposure latitude, good LER and profile shape. The line and space patterns at 75 nm and 80 nm 1:1 duty ratio showed no standing waves, no footing/scum and good collapse margin, indicating the good lithographic performance of the bottom anti-reflective coating.

### Lithography Example 7

A silicon substrate coated with a bottom antireflective coating (B.A.R.C.) was prepared by spin coating the bottom anti-reflective coating solution of Formulation Example 12 onto the silicon substrate and baking at 220° C. for 60 sec. The optimum B.A.R.C film thickness was 78 nm, which was simulated and determined using PROLITH (v.9.3.5). AZ photoresist (T85531; available from AZ Electronic Materials USA Corp.) was then coated on the B.A.R.C coated silicon substrate. The spin speed was adjusted such that the photoresist film thickness was 150 nm. The coated wafer was then soft baked at 100° C./60 sec, exposed with Nikon 306D 0.85NA & 0.82/0.55 Dipole-Y Illumination using attenuated phase shift mask, post exposure baked at 110° C./60 sec, and developed using a 2.38 weight % aqueous solution of tetramethyl ammonium hydroxide for 30 sec. 75 nm and 80 nm 1:1 line and space patterns were then observed on a scanning electron microscope. The photoresist had very good exposure latitude, good LER and profile shape. The line and space patterns at 75 nm and 80 nm 1:1 duty ratio showed no standing waves, no footing/scum and good collapse margin, indicating the good lithographic performance of the bottom anti-reflective coating.

### Lithography Example 8

A silicon substrate coated with a bottom antireflective coating (B.A.R.C.) was prepared by spin coating the bottom anti-reflective coating solution of Formulation Example 12 onto the silicon substrate and baking at 220° C. for 60 sec. The optimum B.A.R.C film thickness was 35 nm which was simulated and determined using PROLITH (v.9.3.5). A model immersion photoresist was then coated on the B.A.R.C coated silicon substrate. The spin speed was adjusted such that the photoresist film thickness was 110 nm. The coated wafer was then soft baked at 95° C./60 sec, exposed with ASML 1700i 1.20NA & 0.979/0.824 Dipole-40Y Illumination using attenuated phase shift mask, post exposure baked at 90° C./60 sec, and developed using a 2.38 weight % aqueous solution of tetramethyl ammonium hydroxide for 10 sec. 45 nm 1:1 line and space patterns were then observed on a scanning electron microscope. The photoresist had good exposure latitude, good LER and profile shape. The line and space patterns at 45 nm 1:1 duty ratio showed no standing waves, no footing/scum and good collapse margin indicating the good lithographic performance of the bottom anti-reflective coating.

1. An antireflective coating composition comprising;
a) the reaction product of a glycoluril compound with a compound 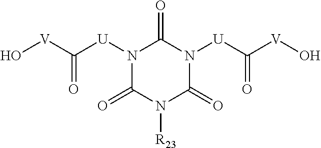
where U is a divalent linking group; V is a direct bond, C₁-C₁₀ straight or branched alkylene, or cycloalkylene group; and R₂₃ is hydrogen or C₁-C₁₀ alkyl.
b) an acid or acid generator.

2. The antireflective coating composition of claim 1, where in step a) the reaction further comprises a polyhydroxy compound.

3. The composition of claim 1 where U is selected from an alkylene group, a phenylene group, a cycloalkylene group.

4. The composition of claim 1 where V is a direct bond.

5. The composition of claim 1, where the compound 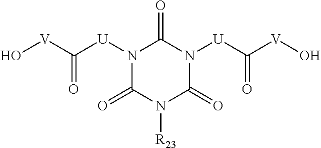
is selected from 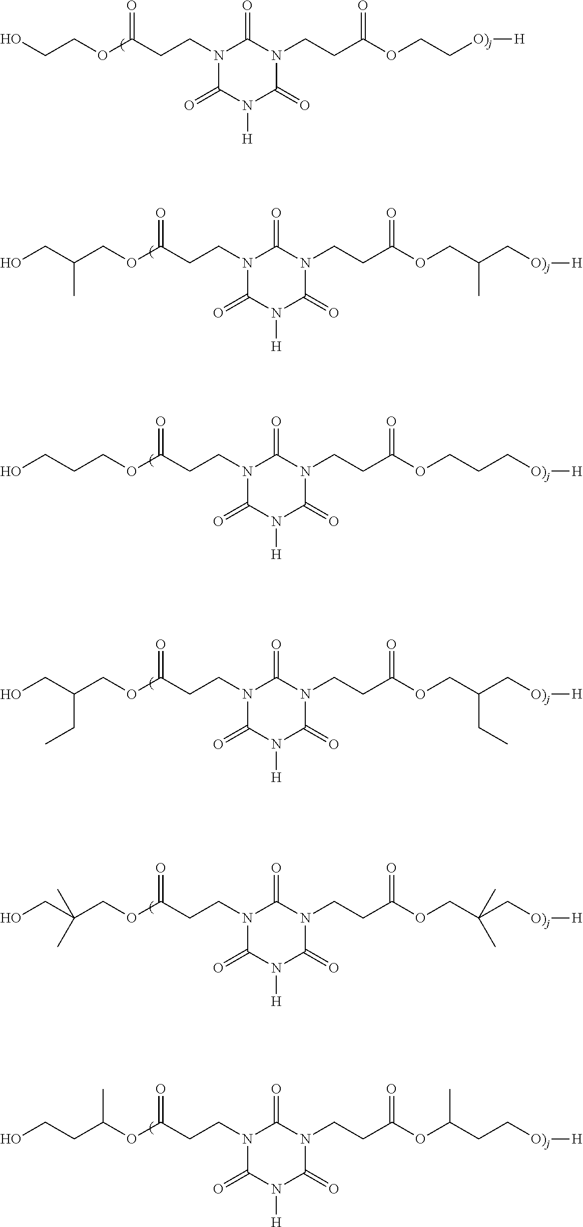 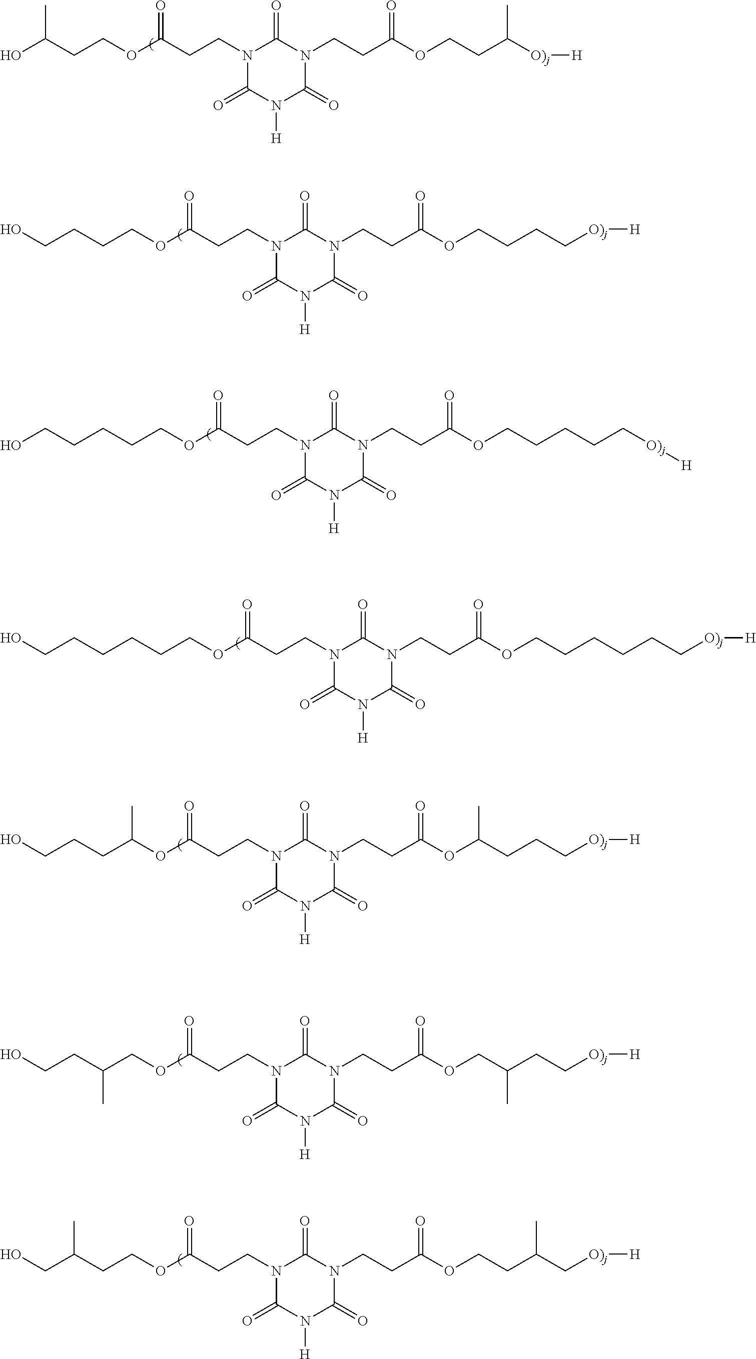 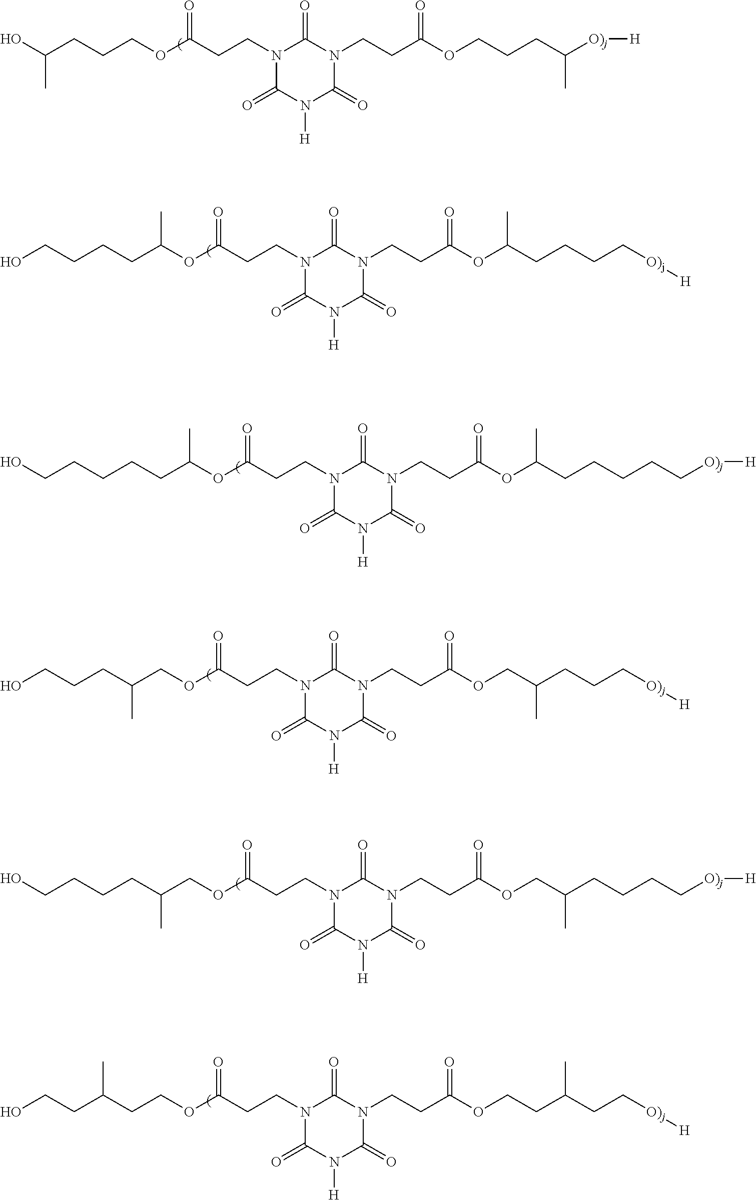 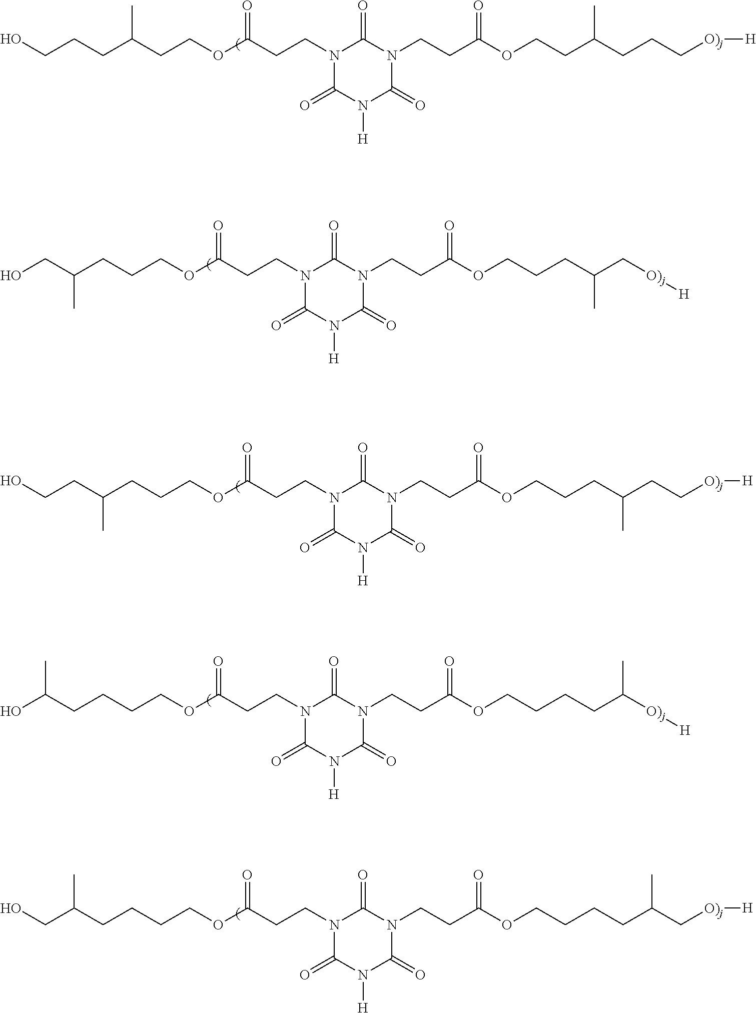 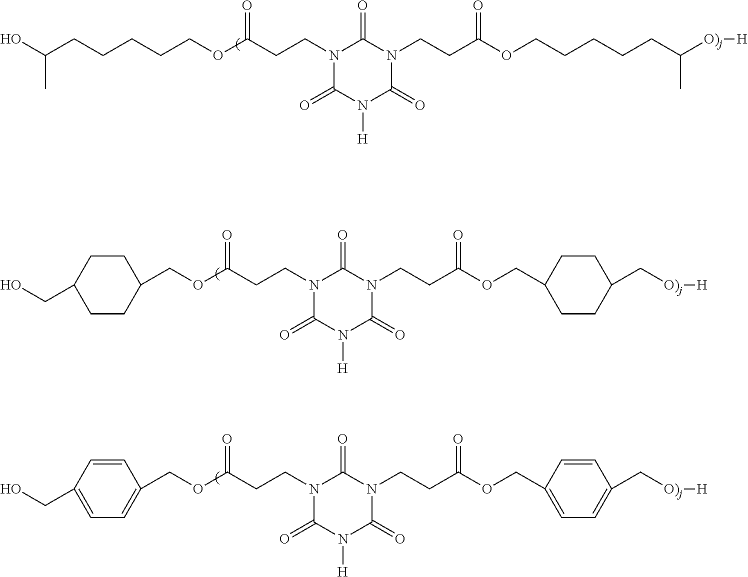
where j is 1 to 5.

6. The composition of claim 1 which further comprises a crosslinker.

7. The composition of claim 6 wherein the crosslinker is selected from glycoluril-aldehyde resins, melamine-aldehyde resins, benzoguanamine-aldehyde resins, urea-aldehyde resins, a compound obtained by reacting a glycoluril compound with a reactive compound containing hydroxy groups and/or acid groups, and mixtures thereof.

8. The composition of claim 6 where the crosslinker is a compound obtained by reacting a glycoluril compound with a reactive compound containing hydroxy groups and/or acid groups.

9. The composition of claim 8, where the reactive compound is selected from ethylene glycol, diethylene glycol, trimethylene glycol, 2,4-dimethyl-2,4-pentanediol, 2,5-dimethyl-2,5-hexanediol, 3-methyl-1,3-butanediol, 3-methyl-2,4-pentanediol, 2-methyl-1,3-propanediol, 2,2-diethyl-1,3-propanediol, 1,3 butanediol, 1,2-butanediol, 2,3-butanediol, 1,2-pentanediol, 2,4-pentanediol, 1,3-pentaediol, 1,4-pentanediol, 1,5-pentanediol, 1,2-hexanediol, 1,6-hexanediol, 2,4-hexanediol, 2,5-hexanediol, propylene glycol, neopentyl glycol, polyethylene glycol, styrene glycol, polypropylene oxide, polyethylene oxide, butylene oxide, 1-phenyl-1,2-ethanediol, 2-bromo-2-nitro-1,3-propanediol, 2-methyl-2-nitro-1,3-propanediol, diethylbis(hydroxymethyl)malonate, hydroquinone, 3,6-dithia-1,8-octanediol, (2,2-bis(4-hydroxyphenyl)propane), 4,4′-isopropylidenebis(2,6-dimethylphenol), bis(4-hydroxyphenyl)methane, 4,4′-sulfonyldephenol, 4,4′-(1,3-phenylenediisopropylidene)bisphenol, 4,4′-(1,4 phenylenediisopropylidene)bisphenol, 4,4′-cyclohexylidenebisphenol, 4,4′-(1-phenylethylidene)bisphenol, 4,4′-ethylidenebisphenol, 2,2-bis(4-hydroxy-3-tert-butylphenyl)propane; 2,2-bis(4-hydroxy-3-methylphenyl)propane, 1,1-bis(4-hydroxyphenyl)ethane; 1,1-bis(4-hydroxyphenyl)isobutane; bis(2-hydroxy-1-naphthyl)methane; 1,5-dihydroxynaphthalene; 1,1-bis(4-hydroxy-3-alkylphenyl)ethane, 2,2-bis(3-sec-butyl-4-hydroxyphenyl)propane, 2,2-bis(4-hydroxy-3-isopropylphenyl)propane, 2,2-bis(4-hydroxyphenyl)butane, α,α′-bis(4-hydroxy-3,5-dimethylphenyl)-1,4-diisopropylbenzene, 2,6-bis(hydroxymethyl)-p-cresol, 2,2′-(1,2-phenylenedioxy)-diethanol, 1,4-benzenedimethanol, phenylsuccinic acid, benzylmalonic acid, 3-phenylglutaric acid 1,4-phenyldiacetic acid, oxalic acid, malonic acid, succinic acid, pyromellitic dianhydride, 3,3′,4,4′-benzophenone-tetracarboxylic dianhydride, naphthalene dianhydride, 2,3,6,7-naphthalenetetracarboxylic acid dianhydride, 1,4,5,8-naphthalenetetracarboxylic acid dianhydride, 3-hydroxyphenylacetic acid, 2-(4-hydroxyphenoxy)propionic acid, a compound (3) obtained by reacting a compound having the formula 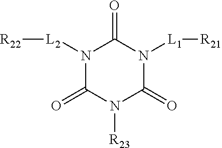
where L₁ and L₂ each independently represent a divalent linking group, R₂₁ and R₂₂ each represent a carbonyl group, and R₂₃ is hydrogen or C₁-C₁₀ alkyl with a polyhydroxy compound,
and mixtures thereof.

10. A compound selected from the group consisting of 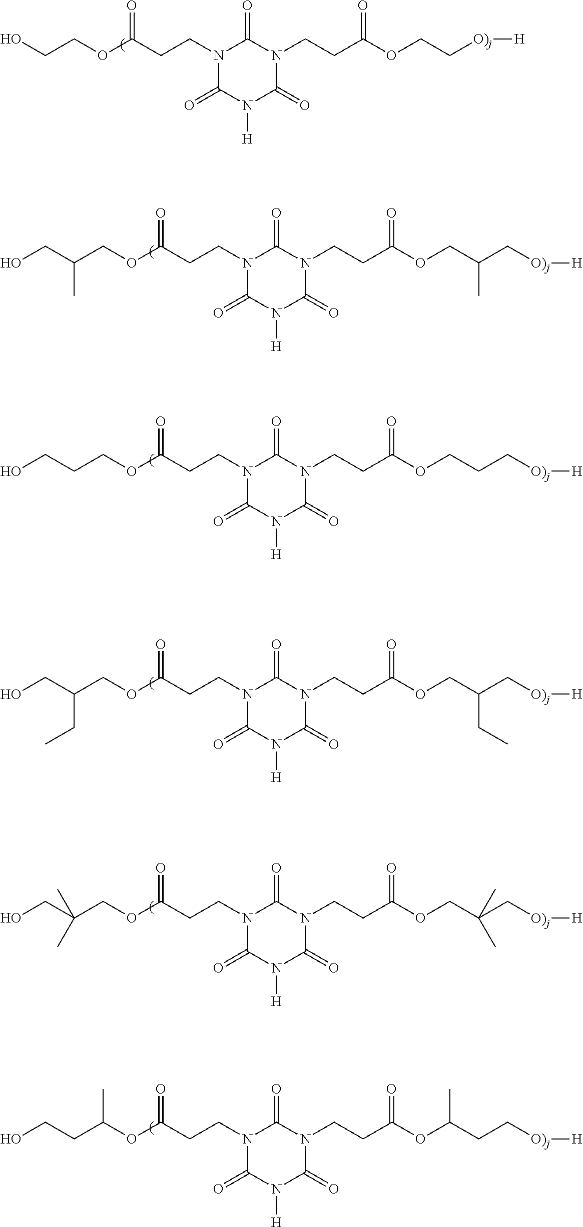 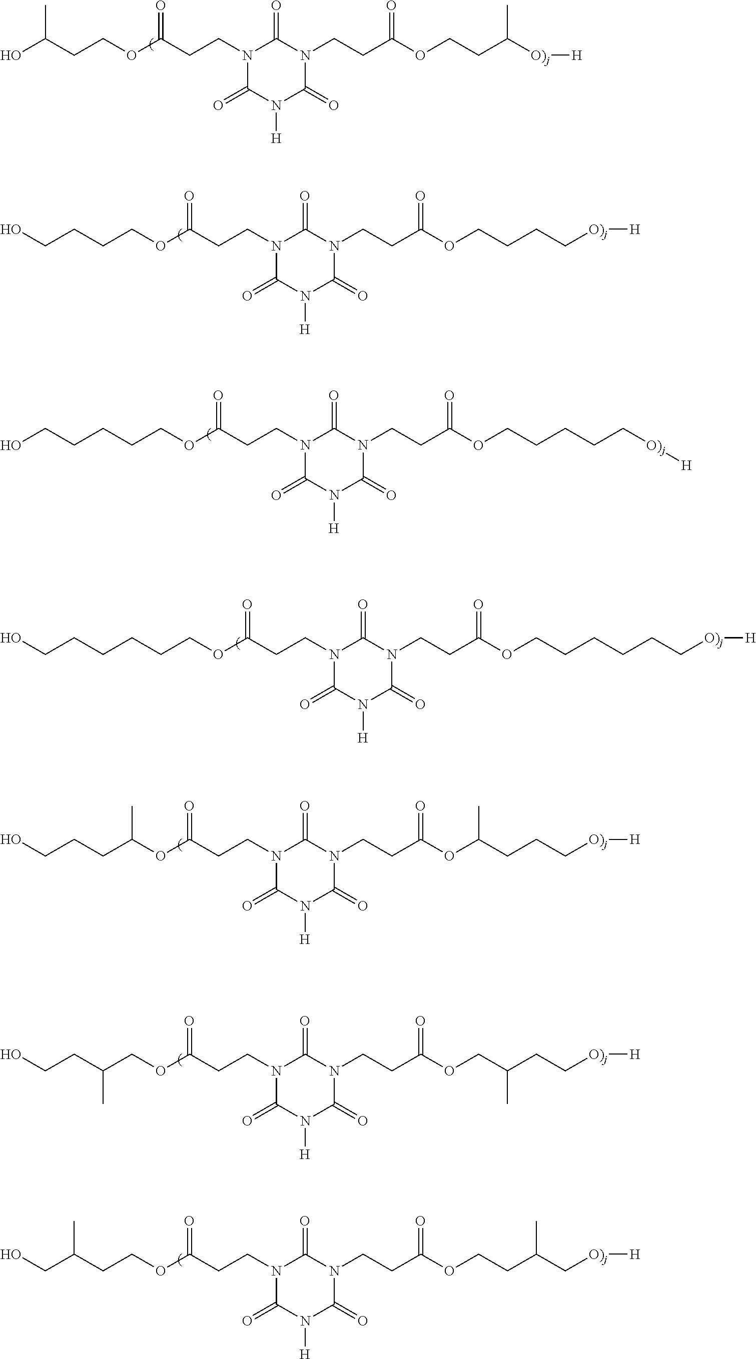 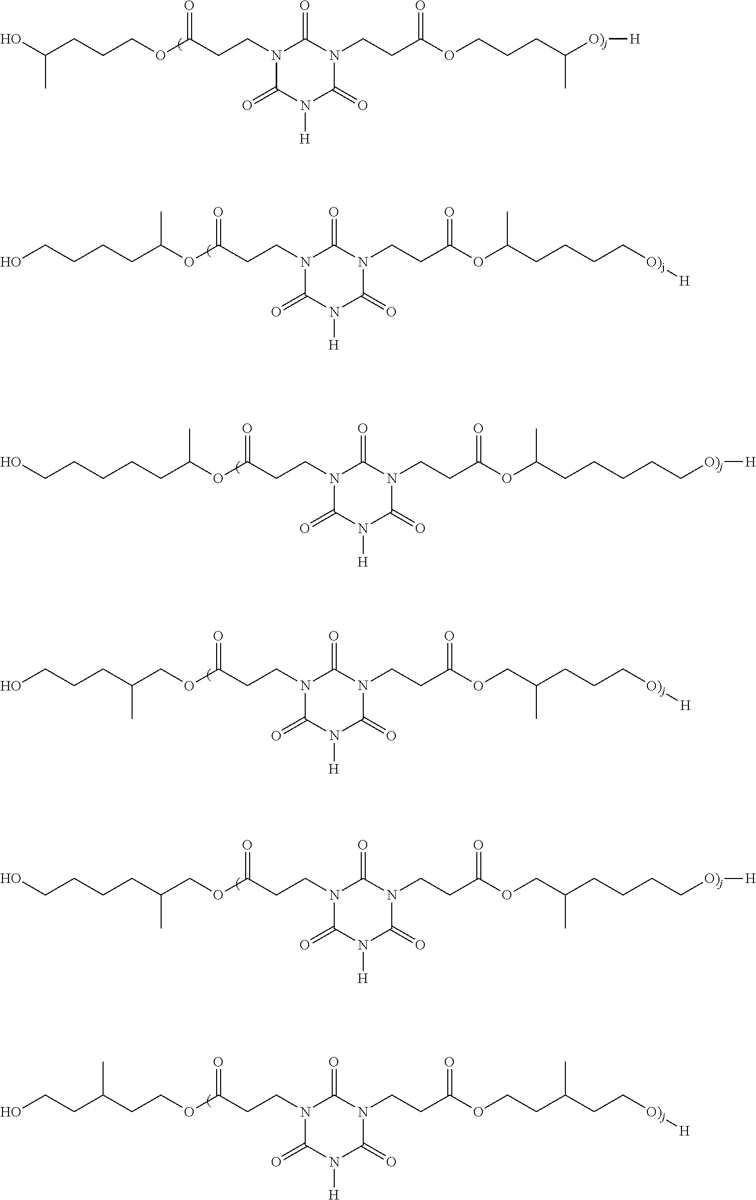 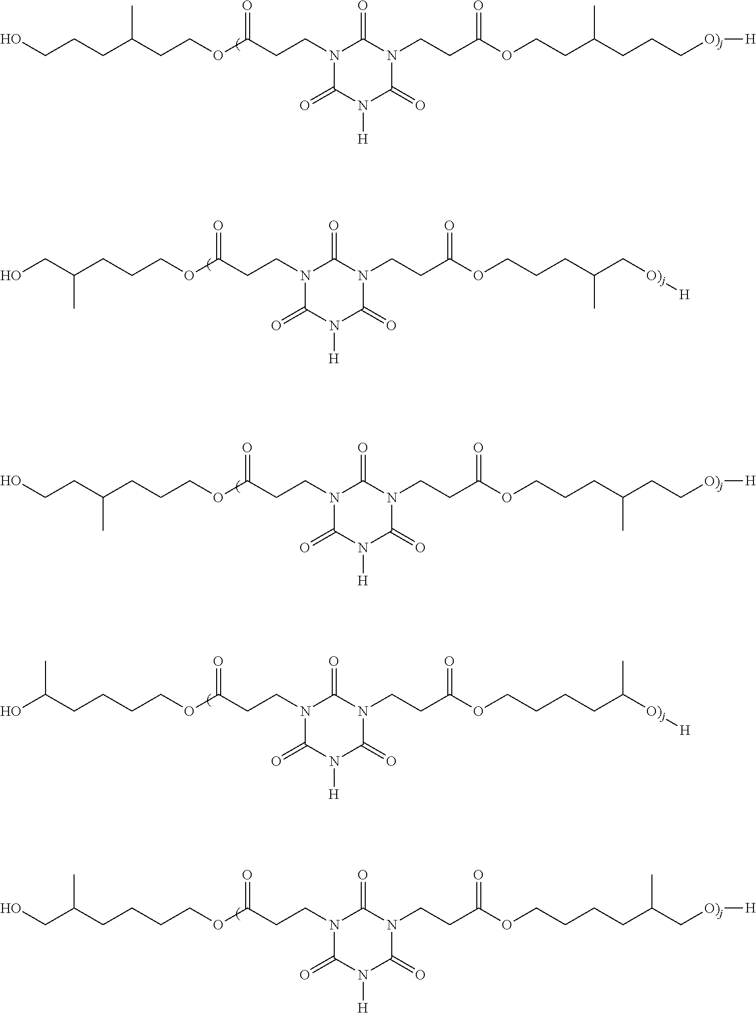 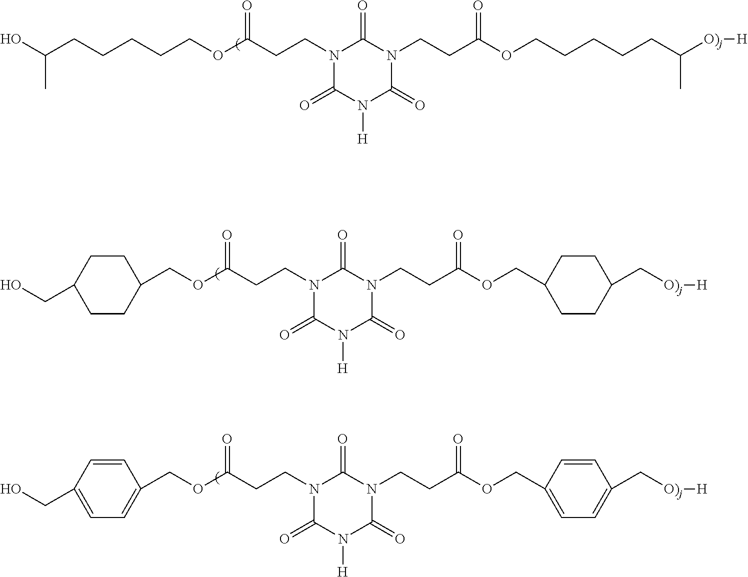
where j is 1 to 5.

11. A process for forming an image comprising, a) coating and baking a substrate with the antireflective coating composition of claim 1; b) coating and baking a photoresist film on top of the antireflective coating; c) imagewise exposing the photoresist; d) developing an image in the photoresist; e) optionally, baking the substrate after the exposing step.

12. The process of claim 11, where the antireflective coating layer has an absorption parameter (k) in the range of 0.01≦k<0.35 when measured at 193 nm.
